(19) United States
(12) Patent Application Publication
(10) Pub. No.: US 2018/0161296 A1
(43) Pub. Date: Jun. 14, 2018

(54) AMINO ACID SUPPLEMENTATION

(71) Applicant: Newcastle Innovation Limited, Callaghan (AU)

(72) Inventors: Richard Hugh Dunstan, Elermore Vale (AU); Timothy Kilgour Roberts, Newcastle (AU); Diane Lisa Sparkes, Rankin Park (AU); Benjamin James Dascombe, Watsonia (AU)

(21) Appl. No.: 15/573,092

(22) PCT Filed: May 11, 2016
(86) PCT No.: PCT/AU2016/050355

§ 371 Date: Nov. 9, 2017

(30) Foreign Application Priority Data
May 11, 2015 (AU) 2015901704

(51) Int. Cl. A61K 31/198; A61K 31/4172; A61K 31/401; A23L 33/175; A61P 43/00

(52) U.S. Cl. CPC A61K 31/198; A61K 31/4172; A61K 31/401; A23L 33/175; A61P 43/00; A23V 2250/0622; A23V 2200/30; A23V 2250/0624; A23V 2250/0642; A23V 2250/0636; A23V 2002/00

(57) ABSTRACT

Provided herein are compositions comprising amino acids and uses of such compositions to aid in recovery from exercise, illness or injury, in performance during exercise, in survival in extreme climatic conditions, and to reduce fatigue. The present disclosure relates to amino acid supplements, which may serve to supplement amino acids lost in sweat.

### FIELD OF THE ART

The present disclosure relates generally to compositions comprising amino acids and uses of such compositions as supplements to supplement amino acids lost in sweat, aiding in recovery from exercise, illness or injury, in performance during exercise, and in survival in extreme climatic conditions.

### BACKGROUND

Amino acids are vital metabolites that are used for the biosynthesis of structural and functional proteins in the body. Depending on the specific roles, the various functional proteins undergo continuous turnover to provide metabolic control and adaptation to physiological demands resulting from exercise, food ingestion, pathogenic challenge and repair of tissue damage. Amino acids also have a wide range of vital roles as “free” metabolites where some can act directly as inhibitory neurotransmitters (e.g. glycine) or act as precursors for the synthesis of hormones (epinephrine and norepinephrine from tyrosine) and neurotransmitters (gamma-aminobutyric acid from glutamic acid).

Following ingestion of food by mammals, proteins are digested and the resultant free amino acids and small peptides are absorbed for utilisation in the body. During exercise, the blood supply is diverted away from the digestive tract to provide oxygen to active muscles and thus the digestion of food cannot be supported. Thus, when the body is subjected to exercise, it compensates for the reduction in freely circulating amino acids by catabolising the non-fibrillar muscle storage proteins, both during and immediately following the exercise. This provides amino acids that can circulate within the blood and be used in metabolic pathways, including in oxidative phosphorylation or the glucose-alanine cycle for energy production.

Due to the utility of free amino acids as a readily oxidisable source of energy and the relevance of certain amino acid residues as indicators of tissue catabolism, measurement of plasma amino acid levels represents a potentially valuable avenue of insight into the muscle condition, protein turnover and energy metabolism of athletes in response to exercise. In healthy resting adults, plasma amino acid levels reflect a tightly regulated homeostasis between nutritional intake and release from tissues, versus tissue uptake and excretion from the body. The onset of exercise represents a disturbance to the resting homeostasis which is required to support the metabolic requirements for the muscles. Alterations in rates of muscle tissue uptake and release of specific amino acids occurs to support demand which results in alterations in the post-exercise plasma profile relative to the pre-exercise profile.

Free amino acids released from tissue into the plasma at the cessation of exercise are theoretically available for re-uptake or excretion and restoration of homeostasis. Amino acids and electrolytes can be leached from the outer stratum corneum of the skin by wetting of the skin surface by sweat and water. This leaching leads to a net increase in concentration of amino acids in the sweat fluid. The amino acid levels excreted in urine and sweat represent a net loss of amino acids, which must ultimately be replenished via dietary intake.

The present invention is predicated on the inventors' findings in relation to combinations of amino acids that predominate in the final sweat with contributions from the skin surface, resulting in the determination of compositions and formulations comprising particular combinations of amino acids to compensate for sweat-facilitated loss of amino acids. Further, the identification of specific profiles or phenotypes for sweat-facilitated loss of amino acids makes it possible to conceive and implement a profile- or phenotype-directed approach to amino acid replenishment which has the benefits of tailoring supplementation to individual needs.

### SUMMARY OF THE DISCLOSURE

In a first aspect, provided herein is an amino acid composition comprising histidine, serine and lysine, wherein the histidine, serine and lysine together comprise at least about 25% of the total weight of amino acids in the composition.

In an embodiment, the histidine, serine and lysine may comprise at least about 30% of the total weight of amino acids in the composition. In another embodiment, the histidine, serine and lysine may comprise between about 30% and 50% of the total weight of amino acids in the composition. In yet another embodiment, the histidine, serine and lysine may comprise between about 32% and 47% of the total weight of amino acids in the composition.

In an exemplary embodiment the histidine may comprise between about 10% to 21%, the serine between about 13% to 16%, and the lysine between about 9% and 10% of the total weight of amino acids in the composition.

The amino acid composition of the first aspect may further comprise at least one of ornithine and glycine. Where present, the omithine may comprise at least about 12%, and/or the glycine may comprise at least about 8%, of the total weight of amino acids in the composition. The composition may comprise histidine, serine, lysine, omithine and glycine, wherein these amino acids comprise at least about 40% of the total weight of amino acids in the composition, or between about 50% and about 80% of the total weight of amino acids in the composition.

The amino acid composition of the first aspect may further comprise at least one of glutamine, glutamic acid, leucine and aspartic acid. Where present, the glutamine and/or glutamic acid may comprise at least about 10%, the leucine at least about 10%, and/or the aspartic acid at least about 7% of the total weight of amino acids in the composition. The composition may comprise histidine, serine, lysine, glutamine and/or glutamic acid, leucine and aspartic acid, wherein these amino acids comprise at least about 35% of the total weight of amino acids in the composition, or between about 40% and about 60% of the total weight of amino acids in the composition.

In an exemplary embodiment, the amino acid composition may comprise serine, glutamic acid, histidine, leucine, lysine, aspartic acid, alanine, glycine, phenylalanine, valine, isoleucine, proline, threonine, and tyrosine. Such a composition may be formulated for administration to horses.

In a second aspect, provided herein is an amino acid composition comprising histidine, serine, omithine, lysine and glycine, wherein the histidine, serine, omithine, lysine and glycine together comprise at least about 30% of the total weight of amino acids in the composition.

In exemplary embodiments the histidine, serine, omithine, lysine and glycine together may comprise at least about 60%, at least about 64% or at least about 76% of the total weight of amino acids in the composition.

In a third aspect, provided herein is an amino acid composition comprising serine, alanine, glycine, histidine and proline, wherein the serine, alanine, glycine, histidine and proline together comprise at least about 20% of the total weight of amino acids in the composition.

In an embodiment, the amino acid composition of the third aspect is formulated for administration to a female subject.

Typically, amino acid compositions disclosed herein are used as dietary supplements. In particular embodiments, the compositions promote or assist recovery from exercise, illness or injury in a subject, reduce fatigue, assist survival of a subject in hot climates, or promote or assist exercise performance.

Amino acid compositions disclosed herein may comprise, consist of or consist essentially of the amino acids specified.

In a fourth aspect, provided herein is a method for promoting or assisting recovery from exercise, illness or injury in a subject, the method comprising administering to the subject an effective amount of an amino acid composition of the first or second aspect.

In a fifth aspect, provided herein is a method for assisting survival of a subject in a hot climate, the method comprising administering to the subject an effective amount of an amino acid composition of the first or second aspect.

In a sixth aspect, provided herein is a method for promoting or assisting exercise performance in a subject, the method comprising administering to the subject an effective amount of an amino acid composition of the first or second aspect.

In a seventh aspect, provided herein is a method for increasing haemoglobin and/or haematocrit levels in the blood of a subject, the method comprising administering to the subject an effective amount of an amino acid composition of the first or second aspect.

In an eighth aspect, provided herein is a method for providing nutritive support to the elderly, the method comprising administering to the subject an effective amount of an amino acid composition of the first or second aspect.

In a ninth aspect, provided herein is a method for reducing fatigue in a subject, the method comprising administering to the subject an effective amount of an amino acid composition of the first or second aspect.

In an embodiment, the subject may be suffering from chronic fatigue. The subject may be suffering from chronic fatigue syndrome. In embodiments in which the subject is female, the amino acid composition to be administered may, for example, comprise at least aspartic acid, asparagine, ornithine and methionine. In embodiments in which the subject is male, the amino acid composition to be administered may, for example, comprise at least serine, alanine, glycine, aspartic acid, valine, proline, tyrosine, asparagine and methionine.

In a method of any one of the fourth to the ninth aspects, the subject may be a human, and the effective amount of the composition to be administered may be between about 50 mg and 10 grams per day.

In a method of any one of the fourth to the ninth aspects, the subject may be a horse, and the effective amount of the composition to be administered may be between about 5 to 50 grams per day.

In a tenth aspect, provided herein is a method for determining a dietary supplement to be administered to a subject, the method comprising:
a) having the subject exercise sufficiently to generate sweat;
b) determining the amino acid composition in said sweat;
c) determining a sweat-facilitated loss of amino acids profile for the subject based on the total amino acid concentration in said sweat, wherein an amino acid concentration of less than about 4,000 μmoles L⁻¹ represents a ‘low’ profile, between about 4,000 and 10,000 μmoles L⁻¹ represents an ‘intermediate’ profile and greater than about 10,000 μmoles L⁻¹ represents a ‘high’ profile;
   wherein stratifying the subject as low, intermediate or high sweat-facilitated loss of amino acids profile determines the quantity (or dosage) of the supplement to be administered, and optionally the quantity or dosage of said supplement to be administered.

In an exemplary embodiment, the sweat is collected from the back of the subject for the determination of amino acid concentration in step c).

In an exemplary embodiment, the determination of a sweat-facilitated loss of amino acids profile for the subject further comprises determining individual amino acid concentrations in the sweat, wherein: (i) the ‘low’ profile is represented by serine, glycine, alanine and histidine comprising about 50% of the amino acids in the sweat, with serine being the major amino acid component of the sweat; (ii) the ‘intermediate’ profile is represented by omithine, serine, histidine and glycine comprising about 70% of the amino acids in the sweat, with ornithine being the major amino acid component of the sweat; and (iii) the ‘high’ profile is represented by histidine, serine, ornithine and glycine comprising about 60% of the amino acids in the sweat, with histidine being the major amino acid component of the sweat.

In an eleventh aspect, provided herein is a method for determining a requirement for dietary supplementation to be administered to a subject, the method comprising:
a) obtaining a plasma sample from the subject; and
b) determining total amino acid composition in said plasma, wherein an amino acid concentration of less than about 2,800 μmoles L⁻¹ represents a ‘low’ operating level of amino acids indicating a need for supplementation.

In accordance with the tenth and eleventh aspects, determination of a ‘low’ sweat-facilitated loss of amino acids profile may indicate an amino acid supplement for the subject comprising serine, glycine, alanine and histidine, wherein the serine, glycine, alanine and histidine together comprise at least about 60% of the total weight of amino acids in the composition.

Determination of an ‘intermediate’ sweat-facilitated loss of amino acids profile may indicate an amino acid supplement for the subject comprising ornithine, serine, histidine and glycine, wherein the ornithine, serine, histidine and glycine together comprise at least about 64% of the total weight of amino acids in the composition.

Determination of a ‘high’ sweat-facilitated loss of amino acids profile may indicate an amino acid supplement for the subject comprising histidine, serine, ornithine and glycine, wherein the histidine, serine, ornithine and glycine together comprise at least about 76% of the total weight of amino acids in the composition.

### BRIEF DESCRIPTION OF THE DRAWINGS

Embodiments of the disclosure are described herein, by way of non-limiting example only, with reference to the following drawings.

FIG. 1. The principle component analysis (PCA) of the log-transformed amino acid concentration data for the concentrations of amino acids in sweat from the combined cohort (n=19). Each athlete was coded for membership within one of three clusters defined as Low (L), Intermediate (I) and High (H) total levels of amino acids measured in the sweat. Each athlete is positioned on the plot according to their corresponding amino acid profile. Athletes from each of the SFLAA groups have been colour coded and it is clear that members of each group are clustered together. This provides evidence that their amino acid composition characteristics are similar within the groups defined by their SFLAA.

FIG. 2. Comparison of the relative percent abundances of amino acids in sweat from the Low, Intermediate and High SFLAA clusters with the corresponding composition of plasma amino acids. For each amino acid: front bar=plasma; second bar=sweat ‘low’ SFLAA; third bar=sweat ‘intermediate’ SFLAA; fourth (back) bar=‘high’ SFLAA. The plasma levels did not alter between subjects from either group, with alanine, glutamine, valine and proline present as major constituents. The composition of sweat collected from the body surface show differential patterns of sweat composition with serine as the major component for the “low” SFLAA cluster, ornithine the major component for the “intermediate” SFLAA cluster and histidine the major component for the “high” SFLAA cluster.

FIG. 3. Total amino acid concentration in sweat following exercise compared with amino acid levels obtained from a washing of the skin surface 12 hours later after the subject had showered and rested overnight at 18-24° C. The subject then showered, dried and a third sample obtained by washing the freshly dries skin surface to demonstrate that amino acids can be leached from the stratum corneum by wetting of the skin surface. Three sample collections were used to generate data with a week between each collection. These results support the concept that amino acids are present as a significant part of the skin's natural moisturising factor and can be leached from the surface by simple addition of water.

FIG. 4. Comparison of the percent relative abundances of amino acids in (a) post-exercise sweat (front bar) with (b) levels observed for a sample taken after 12 hours rest following the post-exercise shower (second bar) and (c) immediately after showering and drying (third or back bar). Values are averages from three separate sampling events from one male participant. The similarity in amino acid composition with the sweat taken following the exercise, mirrors the composition profile with surface washings from the skin immediately after cleaning and drying the surface. The similarity is strong evidence to support the leaching process as a major contributor to the loss of amino acid by the wetting of the skin by the sweat. Combined, the leachate and the quantities excreted in sweat amount to considerable potential losses during exercise.

FIG. 5. (A) The principle component analysis (PCA) plotted from the relative abundances of amino acids measured in in sweat from 47 healthy subjects and 7 subjects suffering from chronic fatigue. Each case was coded for membership within one of four clusters defined by K-means clustering which partitions the subjects into groups to minimise variance within groups and maximise differences between groups. The results from the PCA analysis confirmed the K-means clustering approach by clearly separating members from within each group on the plot. The subjects suffering chronic fatigue were present as members of either group 1 or group 3. (B) The PCA loadings for factor 1 and factor 2 indicating the contributions of the amino acids to the cluster separations.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The term “subject” as used herein refers to any mammal, including, but not limited to, humans, performance animals (such as thoroughbred and other racehorses), livestock and other farm animals (such as cattle, goats, sheep, horses, pigs and chickens), companion animals (such as cats and dogs) and laboratory test animals.

As used herein, the term “effective amount” refers to an amount of a composition or supplement that is sufficient to effect one or more beneficial or desired outcomes. An “effective amount” can be provided in one or more administrations. The exact amount required will vary depending on factors such as the identity and number of individual probiotic strains employed, the subject being treated, the nature of the disease(s) or condition(s) suffered by the subject that is to be treated and the age and general health of the subject, and the form in which the composition is administered. For any given case, an appropriate “effective amount” may be determined by one of ordinary skill in the art using only routine experimentation.

The term “exercise” as used herein refers to any physical exercise by an individual comprising exertion sufficient to generate sweat. As used herein the term “exercise” includes any sporting activity, whether by way of training or formal participation in a sporting endeavour, activity or event. The terms “exercise” and “sport” or “sports” may be used interchangeably herein.

The term “recovery” as used herein in relation to recovery from exercise may include improved recovery times following amino acid supplementation in accordance with the invention as compared to the absence of supplementation. A variety of parameters, including physiological (e.g. blood oxygen levels, heart rate, haemoglobin levels, haematocrit levels), behavioural and observational, are known in the art for determining and assessing recovery times.

The term “performance” as used herein in relation to exercise or sport refers to any parameter of performance appropriate to the exercise or sport being undertaken, including for example strength, speed and/or endurance. Enhanced performance may also be manifested by the ability to overcome muscle fatigue, the ability to maintain activity for longer periods of time, improved efficiency of training or athletic activity, or maintenance or development of muscle mass.

The term “hot climate” as used herein means any climate in which the heat during at least a part of the year is sufficient to cause discomfort to an individual and cause the subject to sweat such that sweat facilitated loss of amino acids occurs. By way of example, the climate may experience temperatures in excess of 24° C., 30° C., 35° C., 40° C. or 45° C.

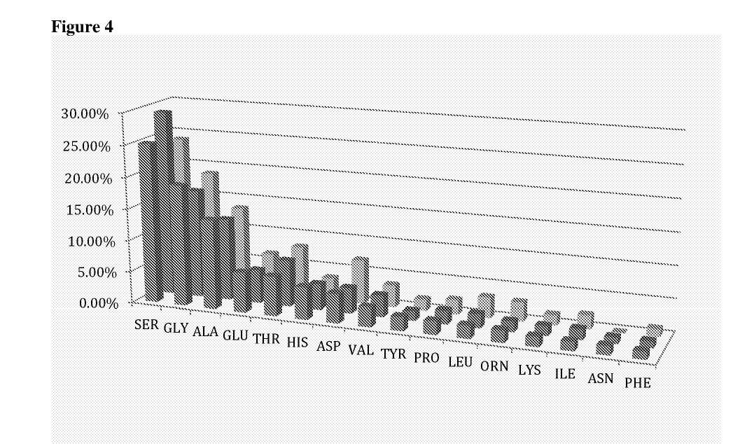

There is potential for individuals to lose significant quantities of amino acids through sweat during exercise, in hot conditions or climates or other periods of physical exertion. When a subject exercises amino acids are removed from the circulating plasma as they are utilised for metabolism to support the exercise. On wetting of the skin by the sweat, the fluid can further leach amino acids from the outer stratum corneum which produces a natural moisturising factor with an amino acid composition similar to the profile of major components measured in the sweat fluid collected from the skin surface (see FIG. 4). To compensate for this, the amino acids would normally be drawn from the non-fibrillar muscle reserves. However, if a subject is subjected to high intensity exercise or training, or over-training, this could result in depletion of non-myofibrillar protein stores, where the body then has no choice but to switch to fibrillar catabolism involving the proteolysis of the structural components actin and myosin with resultant muscle damage, soreness and peripheral fatigue (see, e.g., Niblett et al., 2007; Macintosh and Rassier, 2002). Accordingly, described herein are amino acid supplements, that do not require digestion, for ingestion during or immediately after exercise with a view to delivering amino acids directly to the circulation to minimise potential demands on the muscle protein stores during recovery. This strategy was designed to minimise the impact on the catabolic response in the muscle tissues that continues after exercise has finished as an important period of recovery.

Similarly, patients with ongoing chronic fatigue or a chronic illness with accompanying impaired digestive function may lose amino acids via sweat and urine leading to a sustained catabolic process to meet the body's demand for amino acids. Individuals living in hot climates may also be susceptible to experiencing adverse effects from periodic depletion of amino acids during relatively low levels of exercise or activity, yet which elicit high volumes of continual sweating.

In the human and equine studies described herein, the profile of sweat facilitated losses of amino acids relative to corresponding levels in plasma indicated that the sweat was not merely reflective of plasma amino acid composition. Without wishing to be bound by theory, the data suggest that mechanisms are in place to either facilitate concentration of certain amino acids in sweat by, for example, leaching of amino acids from the skin surface. As exemplified herein, the provision of an amino acid supplement with the amino acids identified as key loss components in sweat is able to raise the plasma amino acid concentrations to levels that may represent a maximal loading of plasma amino acids during work. Again, without wishing to be bound by theory, the inventors suggest that increasing the plasma concentrations of amino acids makes more substrate available for supporting the exercise and recovery whilst reducing demand on muscle stores. As exemplified herein, amino acid supplementation in accordance with the present disclosure has been shown to elevate haemoglobin and haematocrit levels.

The ability to determine sweat-facilitated loss of amino acid ‘phenotypes’ or profiles as described and exemplified herein, find application in the identification of those in need of amino acid support under high intensity exercise, regimes of chronic ill health, chronic fatigue or exposure to hot conditions, and may assist in determining those most suitable to survive most effectively under physically demanding conditions of exercise, training and extreme climatic conditions.

The ability to determine sweat-facilitated loss of amino acid ‘phenotypes’ or profiles as described and exemplified herein facilitates the development of amino acids supplement formulations specifically designed based on gender. Thus, provided herein are amino acid compositions specifically designed for consumption by male human subjects. In particular, amino acid supplementations formulated for male human subjects may comprise_one or more of the amino acids selected from the group consisting of α-amino-adipic acid, asparagine, aspartic acid, glutamine, glutamic acid, glycine, hydroxylysine, histidine, isoleucine, lysine, ornithine, phenylalanine and serine. For a subgroup of males, an amino acid supplement may comprise, for example, histidine, serine, ornithine and glycine as major amino acid constituents. For another subgroup of males, a supplement may comprise, for example, serine, glycine, alanine and histidine as major amino acid constituents. Supplements for males may further comprise, inter alia, glutamine and/or glutamine, proline, serine, glycine, alanine, histidine, ornithine and/or lysine.

Also provided herein are amino acid compositions specifically designed for consumption by female human subjects. In particular, amino acid compositions specifically designed for consumption by female human subjects may comprise one or more of the group consisting of serine, alanine, glycine, histidine, aspartic acid, threonine, glutamine and/or glutamic acid, valine proline, tyrosine and asparagine. Supplements for females may further comprise, for example, ornithine, methionine, cysteine, methionine. Particular supplements may comprise, for example, serine, alanine, glycine, histidine, proline, aspartic acid; asparagine, ornithine and methionine; cysteine and methionine as major amino acid constituents.

Also provided herein are amino acid supplement formulations specifically designed for individuals with chronic fatigue, such as chronic fatigue syndrome. Such supplements for female chronic fatigue subjects may comprise aspartic acid, asparagine, ornithine and/or methionine as the major amino acids in the composition. Supplements for male chronic fatigue subjects may comprise serine, alanine, glycine, aspartic acid, valine, proline, tyrosine, asparagine, and/or methionine as major amino acid components.

Accordingly, the methods and compositions described herein find application in the assessment or evaluation of, and in the provision of supplements for, individuals in a range of environments, professions and industries, promoting or assisting in recovery from exercise or other forms of physical exertion and/or improving performance in said exercise or physical exertion. In accordance with the present disclosure, compositions may be administered to subjects in need before, during or after the exercise or other physical exertion. Suitable individuals may be, for example, athletes (professional, semi-professional or amateur), personal trainers or those undergoing fitness or weight loss programs, military personnel, police and other security workers, firefighters, workers in the construction, mining and related industries, farm workers and stockmen. Those skilled in the art will recognise that this is merely an exemplary list of suitable individuals and the present invention is not intended to be so limited.

As noted above, the methods and compositions described herein also find application in the assessment or evaluation of, and in the provision of supplements for, those experiencing chronic ill health such as, for example, chronic fatigue syndrome, immune deficiencies, those suffering trauma or other injury, and those with impaired digestive function. In accordance with the present disclosure, compositions may be administered to subjects in need before, during or after suffering from the illness, trauma, injury or digestive impairment. One skilled in the art will recognise that digestive efficiency diminishes with age. A further application of the methods and compositions described herein is therefore in the provision of nutritive support to the elderly.

As those skilled in the art will appreciate, the methods and compositions described herein also find application in the assessment or evaluation of, and in the provision of supplements for, non-human subjects. Exemplary non-human animals include horses (such as thoroughbred and standardbred race horses, working horses), dogs (such as racing dogs including greyhounds, and working dogs), and other animals living and/or working in hot conditions.

Those skilled in the art will appreciate that the proportions of each amino acid in compositions and supplements disclosed herein may be adjusted to reflect, for example, the relative losses observed for those amino acids in the sweat either of specific individuals or animals, or of groups of individuals or animals (such as, for example, groups of athletes, racehorses etc.). Thus, the present disclosure contemplates the tailoring of compositions and supplements to the needs of specific individuals or animals or groups of individuals or animals. The determination of the loss of amino acids in sweat is described and exemplified herein, and thus the determination of specific formulations for compositions and supplements is well within the capabilities of those skilled in the art, requiring no undue burden of experimentation.

The skilled addressee will also appreciate that compositions and supplements disclosed herein may comprise, consist of, or consist essentially of, the amino acids as described herein.

In one aspect, the present disclosure provides an amino acid composition comprising histidine, serine and lysine, wherein the histidine, serine and lysine together comprise at least about 25% of the total weight of amino acids in the composition. Depending on requirements for particular subjects, for example as may be determined by analysis of sweat-facilitated loss of amino acids in the subject, these amino acids may comprise at least about 30%, 35%, 40%, 45%, 50%, 55%, 60%, 65%, 70%, 75%, 80%, 85% or 90% of the total weight of amino acids in the composition. Depending on requirements for particular subjects, for example as may be determined by analysis of sweat-facilitated loss of amino acids in the subject, these amino acids may comprise between about 30% and 50%, for example about 30%, 31%, 32%, 33%, 34%, 35%, 36%, 37%, 38%, 39%, 40%, 41%, 42%, 43%, 44%, 45%, 46%, 47%, 48%, 49% or 50% of the total amount of amino acids in the composition.

The histidine may comprise between about 10% to 21%, for example about 10%, 11%, 12%, 13%, 14%, 15%, 16%, 17%, 18%, 19%, 20% or 21% of the total amount of amino acids in the composition. The serine between about 13% to 16%, for example about 13%, 14%, 15% or 16% of the total amount of amino acids in the composition. The lysine between about 9% and 10% of the total weight of amino acids in the composition.

The composition may further comprise at least one of ornithine, glycine, glutamine, glutamic acid, leucine and aspartic acid. Where present, the ornithine may comprise at least about 12%, and/or the glycine may comprise at least about 8%, the glutamine and/or glutamic acid may comprise at least about 10%, the leucine at least about 10%, and/or the aspartic acid at least about 7% of the total weight of amino acids in the composition. In an exemplary embodiment the composition comprises histidine, serine, lysine, ornithine and glycine, wherein these amino acids comprise at least about 40%, 45%, 50%, 55%, 60%, 65%, 70%, 75% or 80% of the total weight of amino acids in the composition. In another exemplary embodiment the composition comprises histidine, serine, lysine, glutamine and/or glutamic acid, leucine and aspartic acid, wherein these amino acids comprise at least about 35%, 40%, 45%, 50%, 55% or 60% of the total weight of amino acids in the composition.

In a further aspect an amino acid composition of the present invention comprises histidine, serine, lysine, ornithine and glycine, wherein the histidine, serine, lysine, ornithine and glycine together comprise at least about 30% of the total weight of amino acids in the composition. Depending on requirements for particular subjects, for example as may be determined by analysis of sweat-facilitated loss of amino acids in the subject, these amino acids may comprise at least about 35%, 40%, 45%, 50%, 55%, 60%, 61%, 62%, 63%, 64%, 65%, 66%, 67%, 68%, 69%, 70%, 71%, 72%, 73%, 74%, 75% or 76% of the total weight of amino acids in the composition.

In addition to the amino acids specified, compositions of the present invention may also comprise any one or more other amino acids and those skilled in the art will appreciate that the scope of the present disclosure is not limited by the inclusion of any particular additional amino acids. In one exemplary embodiment, suitable for equine administration, a composition of the present disclosure may comprise histidine, serine, lysine, glutamine and/or glutamic acid, leucine and aspartic acid, together representing about 60% of the total weight of amino acids in the composition, and further comprising alanine, glycine, phenylalanine, valine, isoleucine, proline, threonine, and tyrosine making up the remaining 40% of the total weight of amino acids.

Compositions of the invention may further comprise other suitable nutritional ingredients (such as minerals, vitamins, coenzymes, fatty acids, carbohydrates, proteins or peptides) as well as additional components to activate incidental benefits in terms of recovery or performance, such as ingredients that improve oxygen metabolism, antioxidants, factors which directly or indirectly are related to radical scavengers or improve cardiac function. The amounts of such other components can be any amount that is considered safe for consumption and approved by the acceptable guidelines of the relevant regulatory authorities. One skilled in the art can adjust such amounts to achieve the desired outcome.

Compositions of the invention may also include any suitable additives, carriers, additional therapeutic agents, bioavailability enhancers, side-effect suppressing components, diluents, buffers, flavouring agents, binders, preservatives or other ingredients that are not detrimental to the efficacy of the composition.

Compositions of the invention can be readily manufactured by those skilled in the art using known techniques and processes well known in the pharmaceutical and nutritional and nutraceutical industries and may be suitably formulated for oral administration. Suitable oral dosage forms may include liquids, granules, powders, gels, pastes, soluble sachets, orally soluble forms, capsules, caplets, lozenges, tablets, effervescent tablets, chewable tablets, multi-layer tablets with, for example, time- and/or pH-dependent release, and the like.

Compositions suitable for oral administration may be presented as discrete units each containing a predetermined amount of each component of the composition as, for example, a powder, granules, a gel, as a solution or a suspension in an aqueous liquid or a non-aqueous liquid. The compositions may be conveniently incorporated in a variety of beverages, food products, nutraceutical products, nutritional supplements, food additives, pharmaceuticals and over-the-counter formulations, as exemplified hereinbelow. However those skilled in the art will appreciate that the compositions may be formulated and provided to users in any suitable form known in the art.

The compositions may be conveniently incorporated in a variety of beverage products. Specific examples of suitable types of beverages include, but are not limited to water, carbonated beverages, sports drinks, nutritional beverages, fruit juice, vegetable juice, milk, and other products that are water-based, milk-based, yoghurt-based, other dairy-based, milk-substitute based (such as soy milk or oat milk) or juice-based beverages. The compositions may be provided in powder, granule or other solid form to be added to the beverage by the user, or premixed in the beverage, or may be provided as a concentrated liquid, gel or paste form to be added to a suitable beverage. Alternatively, the composition may be provided to the user in a liquid form, premixed with a suitable beverage. In one exemplary embodiment the composition may be included in a water-based drink (such as a sports drink) at a dose of about 20 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1500 mg or 2000 mg or greater, depending on the exact nature and volume of the drink.

The compositions may also be conveniently incorporated in a variety of food products, nutraceutical products, or food additives. The food product or food additive may be a solid form such as a powder, or a liquid form. Suitable food products may include baked products such as crackers, breads, muffins, rolls, bagels, biscuits, cereals, bars such as muesli bars, health food bars and the like, dressings, sauces, custards, yoghurts, puddings, pre-packaged frozen meals, soups and confectioneries.

In another embodiment, the compositions may simply be consumed as a powder, granules, gel, paste, solid dosage form or concentrated liquid form in the absence of an additional beverage or food product. Other solid dosage forms are also contemplated, such as capsules and tablets. For example, where the subject is an animal such as horse, the amino acids may be pre-mixed in the appropriate proportions and combined with liquid (such as water) at a suitable ratio with a binder such as xanthan gum to assist in forming a paste delivery system for administration via oral syringe. Those skilled in the art will appreciate that many other oral delivery systems may be employed depending on the identity and tolerances of the subject.

When the composition is formulated as capsules, the components of the composition may be formulated with one or more pharmaceutically acceptable carriers such as starch, lactose, microcrystalline cellulose and/or silicon dioxide. Additional ingredients may include lubricants such as magnesium stearate and/or calcium stearate. The capsules may optionally be coated, for example, with a film coating or an enteric coating and/or may be formulated so as to provide slow or controlled release of the composition therein.

Tablets may be prepared by compression or moulding, optionally with one or more accessory ingredients. Compressed tablets may be prepared by compressing in a suitable machine the components of the composition in a free-flowing form such as a powder or granules, optionally mixed with a binder, lubricant (for example magnesium stearate or calcium stearate), inert diluent or a surface active/dispersing agent. Moulded tablets may be made by moulding a mixture of the powdered composition moistened with an inert liquid diluent, in a suitable machine. The tablets may optionally be coated, for example, with a film coating or an enteric coating and/or may be formulated so as to provide slow or controlled release of the composition therein.

Those skilled in the art will appreciate that single or multiple administrations of compositions disclosed herein can be carried out with dose levels and dosing regimes being determined as required depending on the need of the subject and on the condition of the subject to be treated. The skilled addressee can readily determine suitable dosage regimes. A broad range of doses may be applicable. Dosage regimens may be adjusted to provide the optimum response. Those skilled in the art will appreciate that the exact amounts and rates of administration will depend on a number of factors such as the particular composition being administered including the form in which the composition is administered, the age, body weight, general health, sex and dietary requirements of the subject, as well as any drugs or agents used in combination or coincidental with the compositions. For example, several divided doses may be administered hourly, daily, weekly, monthly or at other suitable time intervals or the dose may be proportionally reduced as indicated by the exigencies of the situation. Based on the teaching herein those skilled in the art will, by routine trial and experimentation, be capable of determining suitable dosage regimes on a case-by-case basis.

In general, compositions of the present disclosure may be administered in any suitable dose amount that is effective as a health supplement, food supplement, food additive, and/or therapeutic agent to achieve the desired health outcome.

In some embodiments, where the subject is human, an effective dose may be in a range of from about 50 mg to 15 g, about 100 mg to 15 g, about 200 mg to 15 g, about 400 mg to 15 g, about 600 mg to 15 g, about 800 mg to 15 g, about 1000 mg to 15 g, about 2 g to 15 g, about 3 g to 15 g, about 4 g to 15 g, about 5 g to 15 g, about 6 g to 15 g, about 7 g to 15 g, about 8 g to 15 g, about 9 g to 15 g, about 10 g to 15 g, about 11 g to 15 g, about 12 g to 15 g, about 13 g to 15 g, or about 14 g to 15 g. An effective dose may be in a range of from about 50 mg to 14 g, about 50 mg to 13 g, about 50 mg to 12 g, about 50 mg to 11 g, about 50 mg to 10 g, about 50 mg to 9 g, about 50 mg to 8 g, about 50 mg to 7 g, about 50 mg to 6 g, about 50 mg to 5 g, about 50 mg to 4 g, about 50 mg to 3 g, about 50 mg to 2 g, about 50 mg to 1000 mg, about 50 mg to 800 mg, about 50 mg to 600 mg, about 50 mg to 400 mg, about 50 mg to 200 mg, or about 50 mg to 100 mg. Such a dose may be administered on a daily or as needed basis. A constant dosage of the composition may be administered over time, for example, about 50 mg per day, about 100 mg per day, about 200 mg per day, about 400 mg per day, about 600 mg per day, about 800 mg per day, about 1000 mg per day, about 1200 mg per day, about 1400 mg per day, about 1600 mg per day, about 1800 mg per day, about 2 g per day, about 2.2 g per day, about 2.4 g per day, about 2.6 g per day, about 2.8 g per day, about 3 g per day, about 3.2 g per day, about 3.4 g per day, about 3.6 g per day, about 3.8 g per day, about 4 g per day, about 4.2 g per day, about 4.4 g per day, about 4.6 g per day, about 4.8 g per day, about 5 g per day up to about 6 g per day, about 7 g per day, about 8 g per day, about 9 g per day, about 10 g per day, about 11 g per day, about 12 g per day, about 13 g per day, about 14 g per day or about 15 g per day, depending on the need of the subject and the form in which the composition is to be administered. Pediatric dosages may be in the range of 15% to 90% of adult dosages.

In some embodiments, where the subject is a horse, an effective dose may be in a range of from about 1 g to 50 g, from about 5 g to 50 g, from about 10 g to 50 g, from about 15 g to 50 g, from about 20 g to 50 g, from about 25 g to 50 g, from about 30 g to 50 g, from about 35 g to 50 g, from about 40 g to 50 g, or from about 45 g to 50 g. An effective dose may be in a range of from about 1 g to 45 g, from about 1 g to 40 g, about 1 g to 35 g, about 1 g to 30 g, about 1 g to 25 g, about 1 g to 20 g, about 1 g to 15 g, about 1 g to 10 g, or about 1 g to 5 g. A constant dosage of the composition may be administered over time, for example, about 1 g per day, about 2 g per day, about 4 g per day, about 6 g per day, about 8 g per day, about 10 g per day, about 12 g per day, about 14 g per day, about 16 g per day, about 18 g per day, about 20 g per day, about 22 g per day, about 24 g per day, about 26 g per day, about 28 g per day, about 30 g per day, about 32 g per day, about 34 g per day, about 36 g per day, about 38 g per day, about 40 g per day, about 42 g per day, about 44 g per day, about 46 g per day, about 48 g per day or about 50 g per day, depending on the need of the equine subject.

In the event that amino acid levels in a subject are at, or have been restored to, normal or acceptable levels, the present disclosure contemplates the administration of compositions disclosed herein in a dose designed to maintain, or assist in maintaining amino acid levels in the subject at normal or acceptable levels. Such a maintenance dose may be lower than a dose required to restore amino acids to a normal or acceptable level or to assist in recovery of a subject, but nonetheless will still typically fall within the range of doses exemplified herein. For example, where the subject is a human, a composition of the present disclosure may be administered to a subject in a dose of about 50 mg per day, about 100 mg per day, about 150 mg per day, about 200 mg per day, about 250 mg per day, about 300 mg per day, about 350 mg per day, about 400 mg per day, about 450 mg per day, about 500 mg per day, about 550 mg per day, about 600 mg per day, about 650 mg per day, about 700 mg per day, about 750 mg per day, about 800 mg per day, about 850 mg per day, about 900 mg per day, about 950 mg per day, about 1000 mg per day, about 2 g per day, up to about 10 g per day in order to maintain acceptable or normal amino acid levels. For example, where the subject is a horse, a composition of the present disclosure may be administered to a subject in a dose of about 1 g per day, 2 g per day, 3 g per day, 4 g per day, 5 g per day, 6 g per day, 7 g per day, 8 g per day, 9 g per day, 10 g per day, 11 g per day, 12 g per day, 13 g per day, 14 g per day, 15 g per day, up to about 30 g per day in order to maintain acceptable or normal amino acid levels. Such maintenance doses may also be suitable, for example, for human athletes or horses outside of exercise, training or competition times or schedules.

The present invention also provides methods for determining the most suitable amino acid constitution for a composition to be administered to a subject, and the most suitable dosage level. Typically such determinations are based on an analysis of the sweat-facilitated loss of amino acids for any given subject and/or the total amino acid concentration in a plasma sample obtained from a subject. For example, as exemplified herein in one embodiment the invention provides a method for determining a dietary supplement to be administered to a subject, the method comprising:
a) having the subject exercise sufficiently to generate sweat;
b) determining the amino acid composition in said sweat;
c) determining a sweat-facilitated loss of amino acids profile for the subject based on the total amino acid concentration in said sweat, wherein an amino acid concentration of less than about 4,000 μmoles L⁻¹ represents a ‘low’ profile, between about 4,000 and 10,000 μmoles L⁻¹ represents an ‘intermediate’ profile and greater than about 10,000 μmoles L⁻¹ represents a ‘high’ profile;
   wherein stratifying the subject as low, intermediate or high sweat-facilitated loss of amino acids profile determines the quantity (or dosage) of the supplement to be administered, and optionally the quantity or dosage of said supplement to be administered.

Stratification subjects as low, intermediate or high sweat-facilitated loss of amino acids profiles may be desirable, for example, in the case of high performance athletes or animals, or in subjects suffering from, or predisposed to serious illness or injury.

Also provided herein is a method for determining a requirement for dietary supplementation to be administered to a subject, the method comprising:
a) obtaining a plasma sample from the subject; and
b) determining total amino acid composition in said plasma, wherein an amino acid concentration of less than about 2,800 μmoles L⁻¹ represents a ‘low’ operating level of amino acids indicating a need for supplementation.

As exemplified herein, the determination of a sweat-facilitated loss of amino acids profile for the subject may further comprise determining individual amino acid concentrations in the sweat, wherein: (i) the ‘low’ profile is represented by serine, glycine, alanine and histidine comprising at least about 50% of the amino acids in the sweat, with serine being the major amino acid component of the sweat; (ii) the ‘intermediate’ profile is represented by omithine, serine, histidine and glycine comprising at least about 70% of the amino acids in the sweat, with ornithine being the major amino acid component of the sweat; and (iii) the ‘high’ profile is represented by histidine, serine, ornithine and glycine comprising at least about 60% of the amino acids in the sweat, with histidine being the major amino acid component of the sweat.

Furthermore, as exemplified herein the present invention contemplates the employment of a blood or plasma test to determine total amino acid levels in the plasma of a subject upon which to determine an amino acid supplement for administration.

The methods described can be used on an ongoing basis to facilitate the development and implementation of a suitable amino acid supplementation program for the subject taking into consideration, for example, current and past performance levels and workload, subject condition, and future requirements. This may involve devising the specific amino acid constitution of the supplement to be administered and/or determining the appropriate dose or doses to be employed at different times.

Compositions and methods of the present disclosure may be employed as an adjunct to other supplement programs, or other therapies or treatments for promoting or assisting recovery from exercise, illness, trauma, or injury or in promoting or assisting exercise or sports performance. Accordingly compositions and methods disclosed herein may be co-administered with other agents that may facilitate a desired outcome. By “co-administered” is meant simultaneous administration in the same formulation or in two different formulations via the same or different routes or sequential administration by the same or different routes. By “sequential” administration is meant a time difference of from seconds, minutes, hours or days between the administration of the agents, compositions or treatments. Sequential administration may be in any order.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the invention.

### EXAMPLES

The following examples are illustrative of the invention and should not be construed as limiting in any way the general nature of the disclosure of the description throughout this specification.

### Example 1—Sweat Facilitated Loss of Amino Acids During Exercise in Male Athletes

The inventors investigated whether significant quantities of amino acids are lost in sweat under defined exercise conditions and constant temperatures and humidity. As described herein below two separate studies were performed under controlled exercise and environmental conditions to generate a total of 19 participants providing sweat for analyses. One study provided 11 subjects with corresponding post exercise blood plasma samples for comparison with the sweat composition of amino acids. The second study provided an extra eight sweat samples to determine whether subgroups of athletes could be delineated based on sweat characteristics such as total amino acid concentrations and fluid losses per hour under exercise regimes at 32-34° C. and 20-30% relative humidity (RH). The studies were approved by the University of Newcastle Human Research Ethics Committee and all participants provided written informed consent prior to inclusion in the study.

### Study Participants

A study group was recruited comprising 11 well-trained male endurance athletes (age: 29±9 yr, height: 179±7, body mass: 73±10 kg, Σ7 skinfolds: 58±24 mm) that had completed at least ten 5 km competitive runs in the past 2 years. Potential participants were excluded if they reported any medical conditions (cardiovascular, musculoskeletal or metabolic) that would have increased their risk of experiencing an adverse event during the exercise. Participants performed three simulated 5 km self-paced time trials on a non-motorised treadmill with various cooling interventions in an environmental chamber to provide a hot environment (32-34° C. and 20-30% RH) separated by seven days. Participants randomly completed the repeat 5 km self-paced time trials where they either underwent pre-cooling by ice slurry ingestion [7.5 g·kg⁻¹·BM⁻¹ of ice slurry (−1° C.) (Gatorade, PepsiCo, New York, USA) in the 30 min prior to the run] mid-cooling by a menthol mouth rinse [swilling 25 mL of an L-menthol solution (0.01% concentration; 22° C.; Mentha Arvensis, New Directions, Sydney, Australia) in the mouth for 5 s prior to expectoration into a bucket], or no intervention.

An additional study group comprising eight male triathletes was recruited to provide sweat samples to facilitate extended subgroup evaluations of sweat composition characteristics. These athletes were aged from 25 to 35 years and had completed an Olympic distance triathlon within the preceding 12 months (age: 29.6±3.4 years, body mass (BM): 77.8±11.1 kg, VO2max: 62.1±4.9 mL kg⁻¹min⁻¹. Olympic distance triathlon time in last 12 months: 2:10:12±0:9:12 h:min:s, mean±SD). Potential participants were excluded if they reported any medical conditions (cardiovascular, musculoskeletal or metabolic) that would have increased their risk of experiencing an adverse event during the exercise. Participants performed two simulated Olympic distance triathlon trials in an environmental chamber to provide a hot environment (32-34° C. and 20-30% RH) separated by seven days. The triathlon comprised three standardised legs including a swim (1500 m) in a 50 m indoor pool, a cycle (1 hour) on a cycle ergometer (Lode Excalibur Sport. Groningen. Netherlands) and a 10 km self-paced time trial on a motorised treadmill (Powerjog JM100, Expert Fitness UK. Mid Glamorgan, Wales). The cycle and run legs where performed within an environmental chamber. Each participant was required to begin the exercise trial hydrated and was weighed just prior to initiating exercise. During the cycle component, participants ingested either 10 g·kgBM⁻¹ of ice slurry (<1° C.) or room temperature (32-34° C.) sports drink (Gatorade, Pepsico, Chatswood, Australia). There was no effect on sweat composition between these cooling strategies.

### Sweat Collection and Analysis

To assess potential contributions from the stratum corneum, sweat and water washings of skin were collected on three separate occasions at weekly intervals. Sweat was collected from the back of one additional male (age 57) following a standardised 30 minute exercise routine for comparison with samples collected from skin water washings. The exercise was undertaken in the early evening at 28-32° C., the participant then showered and slept overnight in temperatures ranging from 18-24° C. Twelve hours post-exercise, a wash sample was collected by spraying filtered water onto the skin of the back sufficient to generate droplets for collection of at least 1 mL in a sterile specimen container. The subject then showered and dried thoroughly before immediately collecting a second sample by spraying and collecting the droplets from the skin. This approach was designed to indicate whether the stratum corneum could contribute amino acids to water on the skin surface as would be expected if leaching had occurred. The project was separately approved by the University of Newcastle Human Research Ethics Committee (approval number: H-2015-0534) and the participant provided written informed consent prior to inclusion in the study.

Plasma samples were taken at the pre- and post-exercise sampling times for the primary study group. The results of multiple plasma samples from each participant at repeat sessions were averaged to provide a single representative value from each individual in the study. Sweat samples were collected during both trials by direct collection into a sterile 70 mL specimen jar (Sarstedt, Germany). In cohort 1, five of the eight athletes provided sweat samples on two occasions; once under conditions of provision of cold slurry and once under provision of ambient temperature fluids. Three of the athletes provided only one sweat sample under provision of either cold slurry or ambient temperature fluids. In cohort 2, each of the 11 athletes provided sweat samples on all three occasions. Sweat was collected by a researcher immediately after the treadmill run by scraping a squeegee over the skin of the middle upper back, triceps and forehead of each participant and immediately transferring the sweat into the sterile container. Results from multiple sweat samples from each participant were averaged to provide a single representative value for each individual in the study. Following the exercise routine, the subjects were dried by towel and weighed to determine total fluid loss during the exercise regime. The total sweat volume was calculated as the total body mass lost throughout the triathlon corrected for fluid and food intake across the simulated triathlon. Sweat samples were kept at 4° C. and were frozen within 60 minutes of collection. The sweat samples were stored at −80° C. until analysis for amino acid composition using the EZ:Faast™ (Phenomenex® Inc.) derivatisation kit for analyses of amino acids by gas chromatography/flame ionisation detection (GC/FID) as previously described by Evans et al., 2008.

### Data and Statistical Analysis

The different cooling treatments had no effects on amino acid composition of plasma or sweat samples as assessed by ANOVA. Replicate sweat and pre- or post-exercise plasma samples for each athlete were thus averaged to include one representative value for each athlete. The datasets were constructed to compare pre- and post-exercise plasma amino acid profiles by one-way ANOVA and levels of statistical significance were set at P<0.05. In addition, plasma and sweat compositions between the groups defined on the basis of total amino acid concentrations in the sweat were also assessed via ANOVA. The sweat excretion clusters were analysed using principal component analysis, discriminant function analysis and correlation analyses using Statistica™ V12 software (Statsoft).

### Results

Twenty-six amino acids were detected in sweat collected from the primary athlete group 1 (n=1). These are summarised in Table 1 for comparison with corresponding plasma amino acids taken at pre- and post-exercise times. Aspartic acid and hydroxylysine were present in the sweat but absent in both pre- and post-exercise plasma samples. The average total concentration of amino acids in sweat was more than three-fold higher than those observed in the blood plasma. A total of 13 amino acids were present at concentrations significantly higher than those recorded in the post-exercise plasma and comprised: α-amino-adipic acid, asparagine, aspartate, glutamic acid, glycine, histidine, hydroxylysine, isoleucine, leucine, lysine, ornithine, phenylalanine and serine. Four amino acids were present in the sweat in significantly lower concentrations compared with the post-exercise plasma and included: α-amino-butyric acid, glutamine, cystine and proline. The post-exercise plasma amino acids showed a statistically significant increase in alanine and significant decreases in asparagine, lysine, ornithine, serine, and threonine. It could therefore be concluded that the exercise regime had an impact on the amino acid composition of the circulating plasma that could not simply be explained, for example, by changes in blood volume.

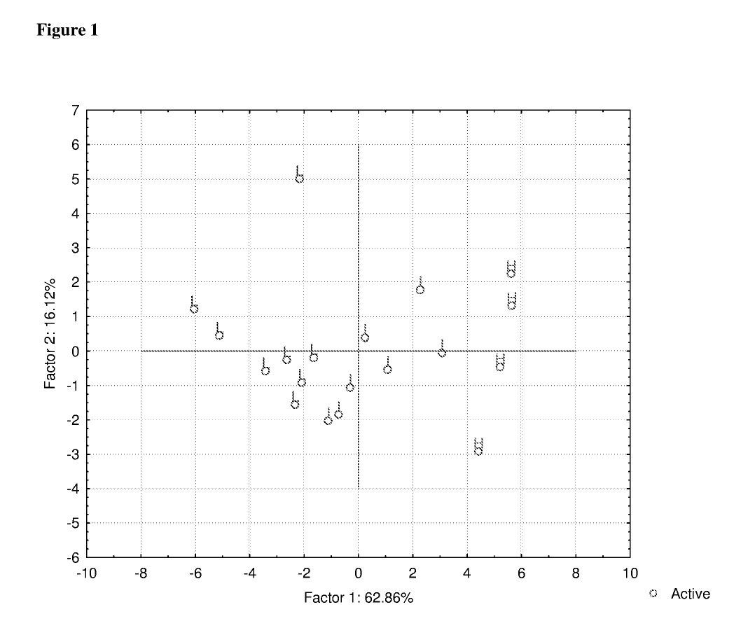

The amino acid composition of sweat was determined for the secondary athlete group (n=8) and compared with the primary group (n=11) data which revealed that there were no significant differences in the total levels of amino acids in sweat between the two groups. Only three amino acids, together representing 5% of the composition of sweat, were statistically different between the primary and secondary study groups. These three amino acids consisted of leucine measured in the primary athlete group at 295±158 μM vs secondary group 106±28 μM; α-amino-adipic acid 74.5±23 μM vs 6.5±0.6 μM; and tyrosine 15.6±7.4 μM vs 127±32 μM (P<0.05). The subjects were thus combined to form a larger dataset (n=19) in an attempt to determine explain the high variances obtained for the sweat amino acid concentrations. The data were appraised for obvious differences in sweating characteristics such as amino acid concentrations, sweat volume and total amino acids lost via sweat. When the individuals were ranked on the basis of their total amino acid concentrations in sweat, it was possible to place the athletes into three distinct clusters or subgroups characterised by their sweat facilitated loss of amino acids (SFLAA): 1) a “Low” cluster was defined as possessing a total amino acid concentration in sweat of <4.0 mM (n=8) with a mean±SD of 2.4±0.7 mM; 2) an “Intermediate” cluster was defined as 4.0 to 10.0 mM (n=7) with a mean of 5.9±1.7 mM; and a “High” cluster was defined as >10.0 mM*(n=4) with a mean of 15.2±3.3 mM. The sweat profiles of amino acid concentrations (excluding the total amino acid levels) were subjected to principle component analysis (PCA) to determine whether the profiles in sweat could be used to objectively differentiate the cluster membership. The analysis presented in FIG. 1 clearly shows that factors generated by PCA fully resolved the members of the three clusters based on the patterns of amino acid composition in the sweat from each individual.

TABLE 1
Comparison of sweat amino acid concentrations with pre- and
post-exercise plasma amino acid levels measured in male athletes

| | Plasma | Plasma | Sweat |
|---|---|---|---|
| | Pre-exercise | Post-exercise | Post-exercise |
| | amino acid | amino acid | amino acid |
| | μM | μM | μM |
| | (mean ± SE) | (mean ± SE) | (mean ± SE) |
| Amino acid | (n = 10) | (n = 10) | (n = 11) |
| α-amino-adipic acid | 1.1 ± 1 | 3 ± 1 | 74 ± 23^{b} |
| α-amino-butyric acid | 15 ± 2 | 12 ± 2 | 4 ± 3^{c} |
| Alanine | 375 ± 23 | 499 ± 23^{a} | 630 ± 123 |
| Asparagine | 39 ± 1 | 32 ± 2^{a} | 62 ± 10^{b} |
| Aspartic acid | 0 | 0 | 174 ± 28^{b} |
| β-amino-isobutyric acid | 1.4 ± 1 | 1.6 ± 1 | 12 ± 7 |
| Cystathionine | 6 ± 2 | 3 ± 2 | 15 ± 12 |
| Cystine^{e2} | 1 ± 1 | 15 ± 2 | 3 ± 2^{c} |
| Glutamine | 430 ± 23 | 380 ± 19 | 73 ± 31^{c} |
| Glutamic acid | 31 ± 3 | 39 ± 3 | 200 ± 32^{b} |
| Glycine | 194 ± 8 | 193 ± 84 | 910 ± 169^{b} |
| Histidine^{e} | 52 ± 3 | 48 ± 2 | 1,400 ± 519^{b} |
| Hydroxylysine | 0 | 0 | 67 ± 26^{b} |
| Hydroxyproline | 4 ± 1 | 2 ± 1 | 3 ± 2 |
| Isoleucine^{e} | 60 ± 3 | 61 ± 2 | 158 ± 34^{b} |
| Leucine^{e} | 119 ± 5.6 | 120 ± 4 | 295 ± 59^{b} |
| Lysine^{e} | 165 ± 8 | 142 ± 6^{a} | 637 ± 211^{b} |
| Methionine^{e} | 18 ± 1 | 19 ± 1 | 24 ± 10 |
| Ornithine | 43 ± 1 | 34 ± 2^{a} | 977 ± 335^{b} |
| Phenylalanine^{e} | 45 ± 1 | 49 ± 2 | 157 ± 37^{b} |
| Proline | 230 ± 15 | 207 ± 9 | 88 ± 10^{c} |
| Serine | 77 ± 5 | 49 ± 4^{a} | 1,240 ± 199^{b} |
| Threonine^{e} | 116 ± 5 | 89 ± 5^{a} | 147 ± 26 |
| Tryptophan^{e} | 38 ± 3 | 31 ± 2 | 104 ± 34 |
| Tyrosine^{e2} | 4.7 ± 3 | 1 ± 1 | 16 ± 7 |
| Valine^{e} | 270 ± 14 | 258 ± 12 | 250 ± 45 |
| Total | 2,350 ± 162 | 2,290 ± 151 | 7,790 ± 1,850 |

^{a}Amino acid levels in the post-exercise plasma were significantly different to pre-exercise plasma levels; Amino acid levels in the sweat were significantly higher ^{b}or lower
^{c}compared with the post-exercise plasma levels;
^{e}Essential amino acids;
^{e2}Tyrosine can be synthesised from phenylalanine and cysteine within cystine can be synthesised from methionine and serine.

The sweat characteristics and amino acid compositions of sweat from each of the three clusters have been summarised in Table 2. The “Low” SFLAA group displayed the highest estimated sweat volume per hour at 2.3 L/h and the lowest total amino acid concentration in sweat at 2.4 μM with an estimated quantity of amino acids lost per hour via sweat at 5.5 mmoles. In contrast, the “High” SLAA group had the lowest estimated sweat volume per hour at 1.5 L/h and the highest amino acid concentration at 15.2 mM with an estimated quantity of amino acids lost via sweat per hour at 22.8 mmoles (Table 3). The “Intermediate” SFLAA group's sweat volume per hour of 1.8 L/h and sweat amino acid concentration of 5.9 mM fell between the “High” and “Low” group values with an estimated quantity of 10.6 mmoles lost via sweat per hour. The amino acids were ranked in order of the component with the highest concentration measured in the sweat from the “Low” cluster, and the differences in amino acid profiles between the three clusters were apparent in terms of concentrations as well as relative abundances in the profiles.

The “Low” SFLAA cluster was characterised by having serine, glycine, alanine and histidine as the four predominant amino acid components comprising 57% of the amino acid composition of the sweat; the “Intermediate” cluster had ornithine, serine, histidine and glycine as the major components comprising 71%; and the “High” cluster had histidine, serine, ornithine and glycine comprising 62% of the sweat amino acid composition. Most of the sweat amino acids for the “Intermediate” and “High” clusters were present at concentrations higher than observed for the plasma but glutamine and proline were always present in lower concentrations in the sweat for all groups. Valine was lower in the sweat for the “Intermediate” and “Low” clusters compared with the plasma, and alanine and tryptophan were also lower in the sweat for the “Low” cluster compared with the plasma. Aspartic acid was not detected in plasma but was present as the sixth most abundant amino acid in the sweat from the “Low” cluster and observed at higher concentrations for the remaining groups. The total amino acids in resting plasma levels were highest in the “Low” cluster and lowest in the “High” cluster, and although the differences were not significant, a strong negative correlation was observed between the resting total plasma concentrations and the total sweat concentrations (r²=−0.99) (ie the higher the amino acid concentration in sweat, the lower the resting concentration of amino acids in plasma).

TABLE 2
Comparison of amino acid concentrations in sweat from the “Low”,
“Intermediate” and “High” SFLAA clusters compared
with the post-exercise composition of plasma.

| | Post-exercise | Sweat amino acid concentrations | | |
|---|---|---|---|---|
| | plasma amino | per SFLAA cluster | | |
| | acid | (μM ± SE) | | |
| | concentrations | | Intermediate | |
| | (μM ± SE) | Low | 4,000 to | High |
| | Primary group | <4 000* | 10,000* | >10,000* |
| Amino acid | (n = 10) | (n = 8) | (n = 7) | (n = 4) |
| Serine | 49 ± 4 | 582^{a} ± 135 | 1,160^{b} ± 111 | 2,410^{d} ± 359 |
| Glycine | 193 ± 84 | 349^{a} ± 36 | 682^{b} ± 78 | 1,590^{c,d} ± 146 |
| Alanine | 499 ± 23 | 235^{a} ± 28 | 457^{b} ± 40 | 1,170^{c,d} ± 73 |
| Histidine^{e} | 48 ± 2 | 212^{a} ± 54 | 1,010^{b} ± 379 | 3,300^{d} ± 986 |
| Ornithine | 34 ± 2 | 151^{a} ± 33 | 1,340^{b} ± 352 | 2,150^{d} ± 530 |
| Aspartate | 0 | 119^{a} ± 18 | 251 ± 33 | 322 ± 130 |
| Threonine^{e} | 89 ± 5 | 117 ± 27 | 212 ± 33 | 250 ± 108 |
| Lysine^{e} | 142 ± 6 | 104 ± 31 | 414^{b} ± 152 | 1,340^{d} ± 352 |
| Valine^{e} | 258 ± 12 | 99^{a} ± 8 | 190^{b} ± 27 | 445^{c,d} ± 24 |
| Leucine^{e} | 120 ± 4 | 87 ± 20 | 212^{b} ± 51 | 477^{d} ± 74 |
| Glutamic acid | 39 ± 3 | 68 ± 23 | 196^{b} ± 24 | 378^{c,d} ± 67 |
| Proline | 207 ± 9 | 59^{a} ± 8 | 93 ± 9 | 156^{d} ± 38 |
| Glutamine | 380 ± 19 | 58^{a} ± 16 | 42 ± 16 | 163 ± 71 |
| Phenylalanine^{e} | 49 ± 2 | 48 ± 10 | 122^{b} ± 29 | 277^{d} ± 41 |
| Isoleucine^{e} | 61 ± 2 | 47 ± 8 | 114^{b} ± 21 | 288^{d} ± 35 |
| Tyrosine^{e2} | 1 ± 1 | 34^{a} ± 13 | 72 ± 36 | 103 ± 59 |
| Asparagine | 32 ± 2 | 20 ± 6 | 60^{b} ± 9 | 114^{c,d} ± 19 |
| Tryptophan^{e} | 31 ± 2 | 11^{a} ± 6 | 52^{b} ± 17 | 215^{d} ± 57 |
| α-aminoadipic acid | 3 ± 1 | 9 ± 5 | 27 ± 10 | 153^{d} ± 36 |
| Hydroxylysine | 0 | 2 ± 1 | 23^{b} ± 10 | 171^{d} ± 36 |
| Hydroxyproline | 2 ± 1 | 0 | 14 ± 6 | 3 ± 2 |
| Total Amino Acid | 2.2 ± 0.15 | 2.4 ± 0.26 | 5.9^{b} ± 0.63 | 15.2^{c,d} ± 1.6 |
| Concentrations in Post- | | | | |
| exercise Plasma and | | | | |
| Sweat (mM) | | | | |
| Resting Total Amino | | 2.45 ± 0.25 | 2.39 ± 0.09 | 2.24 ± 0.11 |
| Acid Concentrations in | | | | |
| Plasma (mM) | | | | |
| Estimated total sweat | Primary Group | 2.3 ± 0.30 (n = 4) | 1.8^{f} ± 0.33 (n = 3) | 1.5^{g} ± 0.1 (n = 3) |
| volume per hour | | | | |
| exercise period, L/hour, | | | | |
| (n = 11) | | | | |
| Estimated total amino | Primary Group | 5.5 | 10.6 | 22.8 |
| acids lost/hour exercise | | | | |
| period, mmoles, (n = 11) | | | | |

^{a}The concentrations of amino acids in sweat for the “Low” cluster were significantly different compared with corresponding levels in the plasma (P < 0.05). The sweat parameters were assessed by Tukey's (HSD for unequal N) where ^{b}Intermediate > Low;
^{c}High > Intermediate;
^{d}High > Low (P < 0.05);
^{f}Intermediate < Low;
^{g}High < Low.
^{e}Essential amino acids;
^{e2}Tyrosine can be synthesised from phenylalanine and cysteine within cystine can be synthesised from methionine and serine.

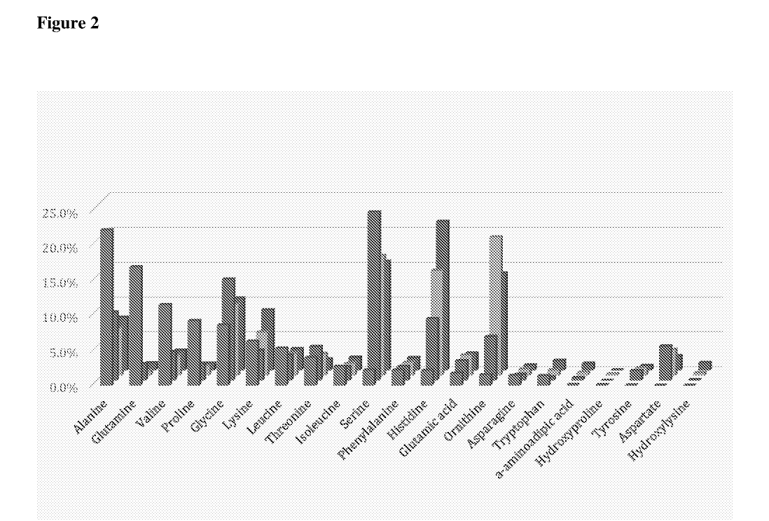

The analyses of the percentage relative abundances of amino acids in the sweat profiles also demonstrated substantial qualitative differences in composition between the three groups. This is illustrated in FIG. 2 where the % relative abundances of amino acids for each of the SFLAA clusters in sweat (FIG. 2A) are plotted for comparison against the corresponding levels in plasma (FIG. 2B). It is apparent from this evaluation that serine, glycine, histidine and ornithine represent the major components in the sweat in all three groups, but each group is characterised by a different amino acid as the dominant component: serine for the “Low” cluster, ornithine for the Intermediate cluster and histidine for the high cluster. Alanine and lysine were also noted for their presence in sweat at levels greater than 5%. In contrast, the major components in the corresponding post-exercise plasma representing 60.1% of the amino acids were alanine at 22.3%, glutamine at 17%, valine at 11.5% and proline 9.3%, all of which were present as Group C amino acids.

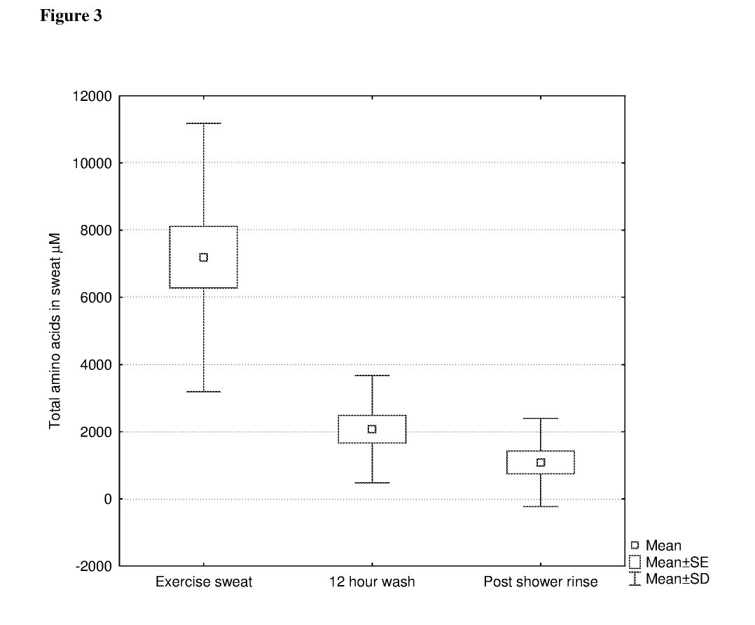

A separate study was undertaken to investigate potential contributions to amino acid loading in sweat from the skin surface itself. The levels of amino acids were measured in sweat following an evening exercise session by one male participant on three separate occasions yielding an average total amino acid concentration of 4.5±1.2 mM. After the exercise collection of sweat, the male then showered and rested overnight for 12 hours at 18-24° C. before collecting a water washing sample by wetting the skin surface of the back with a spray of water and immediately collecting the surface fluid. The surface washing samples taken 12 hours after exercise and showering, were found to have a total amino acid content of 1.2 mM equivalent to 27% of the earlier post-exercise sweat sample. A second water washing sample was taken immediately after showering and drying, again by wetting the skin surface with a spray of water. This sample was found to contain a total amino acid content of around 0.18 mM, equivalent to 4% of the post-exercise sweat sample (FIG. 3). The percentage relative abundances (FIG. 2) for these three types of samples indicated that the amino acid composition profile of the fluid from freshly washed skin surfaces mirrored the composition profile of post-exercise sweat (FIG. 4).

Without wishing to be bound by theory, the inventors suggest that sweating during exercise represents an additional avenue of amino acid loss in comparison to the resting state, over and above amino acid utilisation for energy metabolism, tissue repair and recovery. The process of amino acid loss would involve contributions to the sweat from the skin surfaces by the leaching of amino acids from which occurs on wetting of the skin by sweating. Although the high losses of serine, aspartate, glycine, ornithine and alanine in the current study represented amino acids that can be synthesised by the body, in vivo synthesis of these metabolites during exercise may not necessarily meet demands. Under conditions of prolonged and strenuous exercise, or in hot climates, the losses of amino acids may exceed the body's capacity to access them from non-fibrillar stores or synthesise them de novo. Once these stores are depleted via metabolic oxidation and losses through sweat, the fibrillar proteins may become subject to catabolism to meet the demand for amino acids resulting in potential muscle damage. This form of fibrillar catabolism can result in an inability of the muscle to perform effectively leading to a condition known as peripheral fatigue. Chronic muscle catabolism is a process integral to conditions such as sarcopenia, chronic fatigue, prolonged inactivity and various other disease states as well as accompanying high-intensity exercise or over-training. Although the quantity of amino acids lost through sweat may represent a relatively small proportion of average daily intake, the losses incurred (0.3-1.3 g, or 5.5-22.8 mmoles per hour) would be rapid at a time of high demand when the body's reserves are being utilised via the catabolic response. Based on the World Health Organisation recommended daily allowances, the inventors have calculated that the loss of histidine in sweat during the exercise regime for the “High” SFLAA cluster members may represent up to 40% of their RDA of 10 mg·kg⁻¹·day⁻¹. In a similar way, patients with ongoing chronic illness with accompanying impaired digestive function may lose amino acids via sweat and urine leading to a sustained catabolic process to meet the body's demand for amino acids. People living in hot climates may also be susceptible to experiencing adverse effects from periodic depletion of amino acids during relatively low levels of exercise or activity that elicit high volumes of continual sweating. The method used in the current studies to derive cluster membership may also be employed in future studies to identify those in need of amino acid support under high intensity exercise or regimes of chronic ill health, and it could also help determine those most suitable to survive and exercise effectively under hotter conditions.

### Example 2—Excretion of Amino Acids in Sweat During Exercise Suggests Collagen Turnover is Higher in Adult Human Females than Males

The inventors characterised the amino acid composition of sweat from adults from the general population to assess gender difference in the composition of sweat and whether sweat amino acid patterns of loss represent significant effects on nitrogen balance. Sweat was also measured from a cohort of chronic fatigue subjects to investigate whether subjects with chronic fatigue display higher rates of amino acid excretion resulting in a net negative nitrogen balance.

Participants were adult males and females recruited from the general population as well as a small group of individuals diagnosed with chronic fatigue syndrome (n=7). Sweat was collected from a total of 54 human subjects, comprising 21 females and 33 males. Four females and three males reported suffering from chronic fatigue for more than 3 years. Sweat was collected from the healthy cohort using sterile specimen jars (70 mL, Sarstedt, Germany) gently scraped over the skin surface from their forearms after exercise. Sweat was collected from the fatigued cohort from their forearms which were enclosed in a plastic bag secured below the elbow while sitting at rest in a warm location. Sweat samples were stored at 4° C. and processed within 48 hours of receipt using a commercial kit for analysis by gas chromatography flame ionisation detection (GC-FID; EZ:Faast™). The study was approved by the University of Newcastle Human Ethics committee (H-2014-0086).

The sweat amino acid relative abundance data were arcsine-transformed to improve normality. The data from the CF and healthy groups were combined and subjected to k-means clustering analyses to determine whether discrete groups based on sweat amino acid profiles were discernible. Amino acid concentration data from each of the groups generated by k-means clustering were compared using ANOVA and Tukey HSD for unequal sample sizes. Principle component analysis was performed on the arcsine-transformed amino acid data. All statistics were performed using Dell Statistica version 13 (Dell Inc. 2015).

### Results

Amino acid compositions were determined from the sweat of the 54 subjects. Initial appraisal of individual amino acid levels focussed on comparing the sweat compositions of amino acids from the 30 males and 17 females who were healthy and did not report chronic fatigue. Table 3A summarises the results and indicates the amino acids that had significantly higher concentrations in sweat of females compared with males. No amino acids were significantly higher in males than in females. The total amino acid level in sweat for healthy females was 1.5 times higher than that observed in healthy males (P<0.05). Hydroxyproline, cystine and methionine were 3.8, 2.8 and 2.5 times higher respectively in female sweat than the corresponding levels in male sweat and the other amino acids shown, with the exception of except histidine, were 1.8-2.2 times more concentrated in females than the levels measured for the males (P<0.05). The level of glutamine for females was 95±16 μmoles/L and for males was 79±14 μmoles/L. The percentage relative abundance data also demonstrated significant differences in the amino acids alanine, glycine histidine, proline and hydroxyproline for the healthy male versus the healthy female cohort.

The amino acid composition in sweat for subjects reporting chronic fatigue were compared separately for females and males (Table 3B). With low replicate numbers for the chronic fatigue groups the variances were high but significant differences were apparent. The females with chronic fatigue had generally higher levels of most amino acids measured than healthy females, with significantly higher levels of aspartic acid, asparagine, ornithine and methionine (P<0.05). Males with chronic fatigue had significant increases in the total loading of amino acids in sweat relative to the sweat of healthy males, with specific increases noted in serine, alanine, glycine, aspartic acid, valine, proline, tyrosine, asparagine, hydroxyproline and methionine (P<0.05).

The amino acid concentration data were converted to relative (percentage) abundances and arcsine-transformed for multivariate statistical analyses. K-means clustering analysis revealed that four clusters could be generated with n>6 (Table 4). The females were observed more frequently in cluster 3 with equivalent numbers of males and females observed in cluster 1. Cluster 2 consisted predominantly of males and cluster 4 consisted entirely of males.

The amino acid composition of each cluster was dominated by four major components in each of the clusters which together comprised 57-61% of the amino acids. Serine, glycine, alanine and histidine/aspartic acid were the predominant amino acids in the sweat profiles for clusters 1, 2 and 3. Histidine, serine, ornithine and glycine were the most abundant amino acids for cluster 4 (Table 4).

TABLE 3
Summary of selected amino acid concentrations in the sweat that were significantly
different (a) between males and females, and (b) between healthy subjects
and those reporting chronic fatigue in gender specific groups.
A. Comparisons of amino acids in sweat from healthy males and females

| | | | | | Aspartic | | Glutamic | | |
|---|---|---|---|---|---|---|---|---|---|
| | Serine | Alanine | Glycine | Histidine | acid | Threonine | acid | Valine | Proline |
| Females n = 17 | 2,702 | 1,396 | 1,704 | 653 | 585 | 534 | 491 | 396 | 272 |
| μmoles/L (SE) | (360)* | (155)* | (233)* | (71) | (83)* | (93)* | (80)* | (44)* | (30)* |
| Males n = 30 | 1,337 | 665 | 923 | 944 | 305 | 240 | 256 | 237 | 122 |
| μmoles/L (SE) | (156) | (92) | (117) | (225) | (46) | (28) | (38) | (30) | (16) |
| [symbol] | 114 | 419 | 236 | 89 | 7 | 146 | 60 | 252 | 239 |
| values for | (23) | (89) | (42) | (11) | (4) | (22) | (16) | (37) | (70) |
| plasma‡ μmoles | | | | | | | | | |
| [symbol] | | | | | | | | | |
| Females n = 17 | 26.2% | 13.3% | 16.2% | 6.2% | 5.4% | 5.2% | 4.4% | 3.7% | 2.6% |
| % abundance | (1.3) | (0.5)* | (1.1)* | (0.3)* | (0.5) | (0.5) | (0.3) | (0.2) | (0.1)* |
| Males n = 30 | 21.8% | 9.6% | 13.8% | 11.4% | 5.1% | 4.3% | 3.6% | 3.6% | 2.0% |
| % abundance | (1.5) | (0.4) | (0.5) | (1.5) | (0.5) | (0.4) | (0.3) | (0.1) | (0.2) |

B. Comparisons of healthy subjects vs those reporting chronic fatigue

| | | | | | Aspartic | | Glutamic | | |
|---|---|---|---|---|---|---|---|---|---|
| | Serine | Alanine | Glycine | Histidine | acid | Threonine | acid | Valine | Proline |
| CF Females n = | 3,393 | 1,712 | 2,310 | 553 | 1,216 | 448 | 303 | 588 | 298 |
| 4 μmoles/L | (651) | (330) | (451) | (150) | (414)* | (182) | (79) | (162) | (137) |
| (SE) | | | | | | | | | |
| Females n = 17 | 2,702 | 1,396 | 1,704 | 653 | 585 | 534 | 491 | 396 | 272 |
| μmoles/L (SE) | (360) | (155) | (233) | (71) | (83) | (93) | (80) | (44) | (30) |
| [symbol] | 114 | 419 | 236 | 89 | 7 (4) | 146 | 60 | 252 | 239 |
| values for | (23) | (89) | (42) | (11) | | (22) | (16) | (37) | (70) |
| plasma‡ μmoles | | | | | | | | | |
| [symbol] | | | | | | | | | |
| CF males n = 3 | 3,790 | 1,961 | 2,682 | 560 | 1,055 | 180 | 359 | 592 | 426 |
| μmoles/L (SE) | (865)* | (555)* | (611)* | (288) | (339)* | (180) | (116) | (179)* | (118)* |
| Males n = 30 | 1,337 | 665 | 923 | 944 | 305 | 240 | 256 | 237 | 122 |
| μmoles/L (SE) | (156) | (92) | (117) | (225) | (46) | (28) | (38) | (30) | (16) |

| A. Comparisons of amino acids in sweat from healthy males and females | | | | | | |
|---|---|---|---|---|---|---|
| | Tyrosine | Asparagine | Hydroxyproline | Cystine | Methionine | Totals |
| Females n = 17 | 238 | 170 | 23 | 2.8 | 17.6 | [symbol] 34 |
| μmoles/L (SE) | (27)* | (25)* | (4)* | (0.6)* | (3)* | (1,18 [symbol] |
| Males n = 30 | 108 | 86 | 6 | 1.0 | 6.8 | 6,940 |
| μmoles/L (SE) | (20) | (14) | (2) | (0.4) | (2.1) | (887) |
| [symbol] | 72 | 49 | 20 | 1 | 32 | 3432 |
| values for | (15) | (7) | (11) | (1)^{‡‡} | (6) | (308) |
| plasma‡ μmoles | | | | | | |
| [symbol] | | | | | | |
| Females n = 17 | 2.2% | 1.5% | 0.2% | 0.03% | 0.2% | |
| % abundance | (0.1) | (0.1) | (0.03)* | (0.006) | (0.02) | |
| Males n = 30 | 1.7% | 1.2% | 0.07% | 0.07% | 0.09% | |
| % abundance | (0.2) | (0.1) | (0.02) | (0.004) | (0.02) | |
| B. Comparisons of healthy subjects vs those reporting chronic fatigue | | | | | | |
| | Tyrosine | Asparagine | Hydroxyproline | Ornithine | Methionine | Totals |
| CF Females n = | 350 | 325 | 39 | 942 | 40 | [symbol] 23 |
| 4 μmoles/L | (95) | (100)* | (20) | (204)* | (11)* | (3,10 [symbol] |
| (SE) | | | | | | |
| Females n = 17 | 238 | 170 | 23 | 328 | 18 | [symbol] 34 |
| μmoles/L (SE) | (27) | (25) | (4) | (37) | (3) | (1,18 [symbol] |
| [symbol] | 72 | 49 | 20 | 65 | 32 | 3432 |
| values for | (15) | (7) | (11) | (18) | (6) | (308) |
| plasma‡ μmoles | | | | | | |
| [symbol] | | | | | | |
| CF males n = 3 | 345 | 296 | 41 | 965 | 46 | [symbol] 45 |
| μmoles/L (SE) | (122)* | (121)* | (12)* | (407) | (15)* | (4,16 [symbol] |
| Males n = 30 | 108 | 86 | 6 | 570 | 6.8 | 6,940 |
| μmoles/L (SE) | (20) | (14) | (2) | (142) | (2.1) | (887) |

*P < 0.05;
‡Armstrong and Stave, 1973;
^{‡‡}Dunstan et al, 2016 [symbol]
indicates data missing or illegible when filed

TABLE 4
Comparisons of amino acids in urine, sweat and plasma with calculated losses
based on estimated average daily urinary and sweat excretion rates.

| | | | | | | | |
|---|---|---|---|---|---|---|---|
| | | | | Total | Total* | Total | Total |
| | Urine* | Sweat | Plasma | in 3 L^{‡} | In 1.5 L | in 0.5 L | in 2 L |
| Amino acid | μmoles/L | μmoles/L | μmoles/L | Plasma | Urine | Sweat | Sweat |

Essential amino acids

| | | | | | | | |
|---|---|---|---|---|---|---|---|
| Histidine Males | 1,315 | 944 | 89 | 267 | 1,973 | 472 | 1,888 |
| Females | 1,041 | 653 | | | 1,562 | 327 | 1,306 |
| Lysine Males | 263 | 387 | 198 | 594 | 395 | 194 | 774 |
| Females | 234 | 205 | | | 351 | 103 | 410 |

BCAA

| | | | | | | | |
|---|---|---|---|---|---|---|---|
| Leucine Males | 32 | 235 | 160 | 480 | 48 | 118 | 470 |
| Females | 29 | 250 | | | 44 | 125 | 500 |
| Isoleucine Males | 10 | 145 | 84 | 252 | 15 | 73 | 290 |
| Females | 11 | 188 | | | 17 | 94 | 376 |
| Valine Males | 45 | 259 | 252 | 756 | 68 | 130 | 518 |
| Females | 45 | 396 | | | 68 | 198 | 792 |

Non-essential amino acids

| | | | | | | | |
|---|---|---|---|---|---|---|---|
| Glycine Males | 975 | 923 | 236 | 708 | 1,463 | 462 | 1,846 |
| Females | 1,198 | 1,704 | | | 1,797 | 852 | 3,408 |
| Proline Males | 8 | 122 | 239 | 717 | 11 | 61 | 244 |
| Females | 11 | 272 | | | 16 | 136 | 544 |
| Alanine Males | 264 | 665 | 419 | 1,257 | 396 | 333 | 1,330 |
| Females | 251 | 1,396 | | | 337 | 698 | 2,792 |
| Serine Males | 315 | 1,367 | 114 | 342 | 473 | 684 | 2,734 |
| Females | 360 | 2,702 | | | 540 | 1,351 | 5,404 |
| Aspartic acid Males | 14 | 305 | 7 | 21 | 21 | 153 | 610 |
| Females | 21 | 585 | | | 32 | 293 | 1,170 |
| Glutamine Males | 542 | 78 | 645 | 1,935 | 813 | 39 | 156 |
| Females | 476 | 95 | | | 714 | 48 | 190 |
| Totals Males | 5,185 | 6,940 | 3,432 | 8,886 | 7,778 | 3,470 | 13,880 |
| Females | 4,955 | 10,354 | | | 7,433 | 5,267 | 21,068 |

Cluster 1 was characterised by having the highest concentrations of amino acids in sweat which included significantly higher concentrations of essential amino acids compared with the other clusters (P<0.05). In this same cluster, the elevations of total amino acid loads in sweat were primarily driven by high concentrations of serine, glycine, alanine, aspartic acid, ornithine and histidine. Cluster 3 also had higher levels of glycine, alanine and valine compared with cluster 2. Clusters 1 and 2 had hydroxyproline and proline in the sweat whereas clusters 2 and 4 did not. Cluster 4 was characterised by having the highest concentrations of histidine, lysine and ornithine. Cluster 2 displayed high variances for most of the amino acids and a closer inspection revealed that the males (n=11) had a total amino acid level in sweat of 3,851 (±856) compared with the females at 11,144 (±5,294).

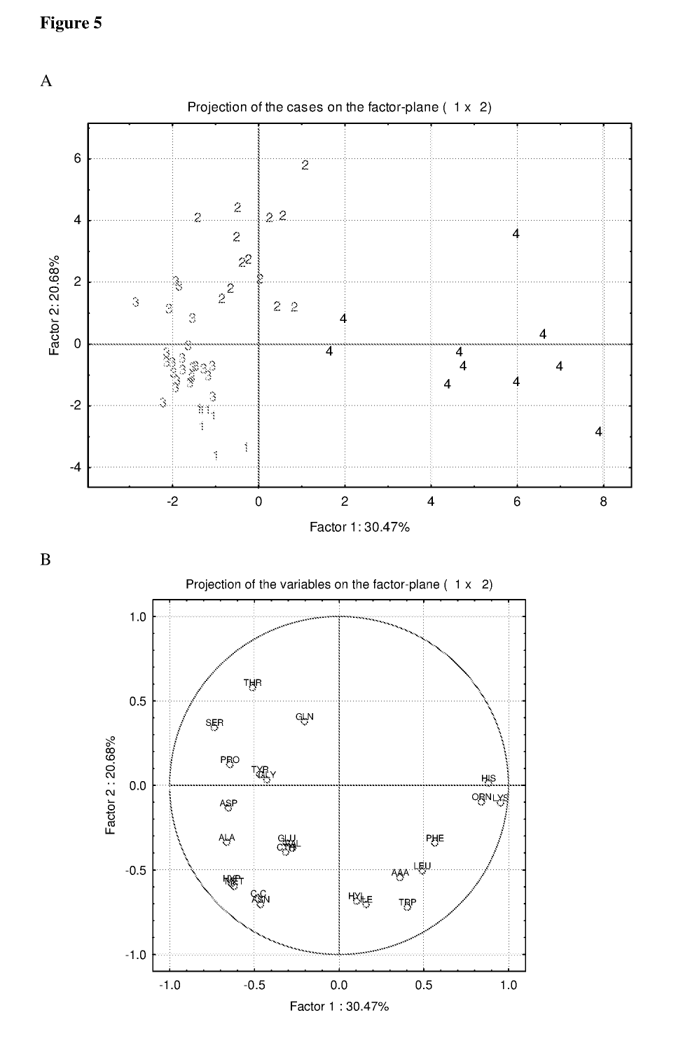

The relative abundance data for the amino acids were subjected to principle component analysis (PCA) and the cases were colour coded based upon group membership as determined by k-means clustering. It was clear from the scatterplot in FIG. 5A that the cases from each cluster were well resolved from each other. The cases in cluster 4 were spread along factor 1 which was aligned with the contributions from histidine, ornithine and lysine as shown in the factor loadings in FIG. 5B.

The concentrations of thirteen amino acids in sweat from healthy females were higher than the corresponding levels measured in males, resulting in a significantly higher total amino acid loading in sweat from females. Glutamic acid (8.1×), histidine (7.3×) and glycine (7.2×) were also substantially concentrated in the sweat relative to plasma. These results are consistent with the proposal that these amino acids may be concentrated in the sweat via a process of leaching of the amino acids from the natural moisturising factor (NMF) found in the skin surface of the stratum corneum

The high concentrations of glycine, serine and alanine in sweat and the potential for high excretion losses represent probable issues for metabolic homeostasis under conditions of high intensity athletic training, prolonged exposures to high temperatures, injury and pathogenic challenge. This is because their losses via sweat may limit maintenance, repair and recovery processes associated with general protein synthesis and cell division. For example, these amino acids, together with proline, are required in relatively high proportions for the synthesis of collagen proteins. The very high levels of sweat facilitated losses of glycine and serine could therefore become limiting for collagen synthesis, especially in females. The concentrations of proline in female sweat were double those measured in males which could indicate a further susceptibility of females to a reduced capacity for collagen synthesis. It may be that males have some capacity to restrict losses of proline via sweat where females do not. The higher levels of proline may simply reflect a higher rate of collagen degradation and subsequent release of proline for females. The excretion of hydroxyproline in females, which is also released during collagen catabolism, was nearly four times that measured in male sweat indicative of higher rates of collagen turnover in the females. Thus a higher rate of collagen turnover coupled with a lower ability to retain proline and glycine, alanine and serine suggested that females may have slower muscle tissue recovery and repair rates.

Females consistently displayed higher rates of amino acid losses in sweat compared with males. Furthermore, individuals with chronic fatigue displayed higher amounts of amino acids in sweat than healthy individuals of the same gender. Glycine and histidine were the major components found in sweat and, without wishing to be bound by theory, the inventors suggest that the amino acids lost in sweat represent limiting components to maintain protein turnover and supporting metabolism, repair and recovery processes. Higher levels (concentration and relative abundance) of proline and hydroxyproline in sweat from females and chronic fatigue subjects pointed to generally higher rates of collagen turnover in these subjects. Glutamine concentrations in sweat were consistently low indicating in that it had most likely undergone deamination in the stratum corneum to produce glutamic acid and pyroglutamate as part of the natural moisturising factor.

Amino acid concentrations in urine were compared to those in sweat and with literature values plasma concentrations (Table 4). In order to estimate average daily losses in urine, a volume of 1,500 mL was taken to represent average daily urinary excretion rates in humans; this allowed for comparisons to be made with total amino acid loading of circulating plasma and of sweat. Sweat rates will vary depending on gender, temperature and humidity and activity levels as well as underlying genetic factors regulating body characteristics such as BMI and fitness levels. Maximum sweating rates have been established at 1-2 L per hour during exercise. These rates have been observed in workers in prolonged hot conditions where 10-12 L of sweat per day can be lost. Training and competition in most sports, in temperatures ranging from 19° C. to 33° C., can generate sweat rates between 0.7 and 2 L per hour for males and females while rowing has been shown to generate 0.8 L per hour at 10° C. It was therefore deemed reasonable to present potential losses in sweat for comparison against urinary output and plasma concentrations for daily volumes of sweat of 0.5 L and 2 L (Table 3). These comparisons revealed that total amino acids losses based on relatively conservative estimates of a daily sweating rates, consistent with no exercise, would result in lower quantities of amino acids being lost in comparison with urinary losses. It was noted that sweat losses were higher in the females compared with the males. These comparisons also demonstrated that not all amino acids are lost equally in urine and sweat. The process of kidney reabsorption is very efficient for the branch chain amino acids and proline, but histidine and glycine were lost in urine at more than four times the concentrations measured in the plasma. In contrast to sweat, where it was proposed the amino acid composition of sweat excreted from the eccrine glands would be similar to that previously seen for plasma but enriched via the leaching of the NMF in the stratum corneum, there is substantial potential for losses of serine, alanine, glycine, histidine and aspartic acid. It was thus concluded that sweating represents a major pathway for losses of those amino acids associate with the function of the NMF in the skin surface.

When the sweat rate was elevated to 2 L to account for inclusion of exercise in the day, the model for losses of amino acids was nearly double that in urine for males and triple that in urine for females. In males 78% of the total loading of the amino acids circulating in plasma is lost for every L of sweat, and in females 116% of the plasma load is lost in 1 L of sweat. This represents a considerable demand on plasma resources of amino acids which must be maintained by the body. To ensure amino acid homeostasis in the plasma whilst food ingestion is not possible exercise, amino acids are derived via proteolysis of non-myofibrillar proteins to provide amino acids for energy, recovery and repair. The requirements for new protein synthesis can remain elevated after exercise for 24-36 hours in athletes and up to 48 hours in the untrained individual. It was thus concluded that females would be more susceptible to developing a net negative nitrogen balance as a result of exercise, exposure to hot climate conditions, poor diet, stress, injury or pathogenic challenge.

### Example 3—Sweat Facilitated Loss of Amino Acids, and Amino Acid Supplementation, in Horses

The inventors characterised the amino acid composition of equine sweat to assess the potential for sweat facilitated losses of amino acids resulting from exercise and investigated the potential for amino acid replacement via supplementation to improve the condition of horses during periods of training.

The study comprised two cohorts of five to six Standardbred harness racing geldings, aged from 3 to 5 years, with no history of significant disease or suffering from any significant ailments or injuries at the beginning of the study. The study was approved by the University of Newcastle Animal Care and Ethics Committee.

### First Cohort

In the first cohort (n=5) all horses followed an identical training schedule under supervision of one trainer, and were actively engaged in competitive racing throughout the course of the study. Sampling was integrated into the regular training regime of the horses and was scheduled to coincide with sessions involving a high intensity track workout. As a result, samples were collected once a week for a period of three weeks, providing a maximum of five pre- and five post-exercise samples per horse. Prior to each training session the horses were fitted with a GPS/heart rate monitoring device to allow for the measurement of speed, distance, effort and recovery. The horses underwent daily training every morning from Monday to Saturday (except on race days) prior to receiving supplementation and feed. The horses received 1 kg Hygain Powatorque (crude protein 17%, crude fat 10%, maximum crude fibre 10%, added sale 1.5%, calcium 1.5%, phosphorous 0.6%, lysing 11 g/kg, vitamin E 1000 IU/kg, selenium 1.5 mg/kg; Hy Gain Feeds Pty Ltd., Victoria, Australia) after the morning workout and again in the evening. The horses also received 2.5-4.5 kg Hygain Microbarley® (crude protein 11%, crude fat 2%, maximum crude fibre 9%) for each animal depending on size and condition. All horses received liberal quantities of wheaten chaff and Lucerne chaff and this feeding regime remained constant throughout the baseline, supplementation and final assessment periods. No other vitamins or supplements were provided during the experimental period. On Sundays, the horses were allowed to forage on grass in the open paddock.

For amino acid supplementation, the horses were provided with a complex amino acid supplement (Fatigue Reviva™; Top Nutrition Pty Ltd) daily for 34 days (with the exception of race day restrictions) before beginning two weeks of post-supplementation samplings and evaluations. The amino acid supplement comprised 20 L-amino acids (glycine, proline, glutamine, carnitine, threonine, lysine, alanine, valine, taurine, serine, cysteine, arginine, histidine, isoleucine, phenylalanine, leucine, methionine, glutamic acid, aspartic acid, and tyrosine), fructo-oligosaccharide, malic acid, citric acid, succinic acid, ribose, and 13 minerals and 13 vitamins. The formulation was provided in a large resealable plastic container and was mixed daily with MCT (mid-chain triglycerides) oil 1:1 to form a paste before oral delivery via 60 mL plastic syringes. The human dosages were adjusted appropriately for horses with 30 g of the Fatigue Reviva™ being provided to each horse daily (except as precluded by racing), which delivered 14.1 g amino acids. After 34 days of supplementation, blood and sweat samples were taken before and after hard work training sessions on three separate occasions over a two week period whilst supplementation was continued.

### Second Cohort

A second cohort of horses was studied with a view to replicating the analyses of sweat composition from the first cohort using a different sample of animals. Again all horses followed the same training schedule under the supervision of the same trainer, and were actively engaged in competitive racing throughout the period of the study. The horses were sampled four times over an initial two week period to obtain baseline measures and were then provided with an amino acid supplement (see below) during training and racing for 64 days. Four sweat and plasma samples were taken from each horse over the last two weeks of the supplement trial. Two horses were withdrawn after stage one baseline testing and prior to supplementation due to injury concerns, and were replaced with another Standardbred horse. This provided six animals for stage one evaluations and four which were then provided with the supplement during training and racing for 40 days. Post-supplementation blood samples were taken before and after hard work training sessions, while sweat samples were taken following training, on four separate occasions over a two week period whilst supplementation was continued. The sample collection periods were extended due to delays resulting from inclement weather. Resting plasma samples were evaluated from a set of seven horses at the property of the trainer and assessed as horses not in work (4 months-7 years) and ranged in age from 3-14 years old (6 geldings and one brood mare). These horses had not been provided with any of the Hygain high protein content feeds for at least four months prior to assessment and were foraging on grass in the paddocks. Because these horses had not received high protein dietary support, they were used as a reference group for comparison on their amino acid levels in plasma.

This second cohort of horses was provided with a supplement (Hygain Omina R3, Hy Gain Feeds Pty Ltd) formulated to contain only the 14 amino acids identified as representing the major amino acids components lost in sweat (serine, glutamic acid, histidine, leucine, lysine, aspartic acid, alanine, glycine, phenylalanine, valine, isoleucine, proline, threonine, and tyrosine). The proportions of amino acids were adjusted to reflect the relative losses observed for the amino acids in the sweat for these animals. These amino acids were pre-mixed in the appropriate proportions and were combined with the Aqa Gel base product 1:1 (HyGain Feeds Pty Ltd) to form a paste delivery system via 60 mL syringes, which was well received by the animals. The paste comprised 30 g amino acids with 500 mg glucose in 30 ml Aqua Gel per serve.

### Experimental Procedures

For both cohorts of horses, the exercise sessions involved two hard work sessions per week when the horses were not raced, or one hard work session and a race. The horses were raced on average once every three weeks. The hard work sessions involved pacing around a 700 m track in full racing harness while pulling a sulky and driver. Each session comprised approximately 2.5 ‘warm up’ laps of the track at a moderate pace, accelerating to racing speed for 3-4 laps, then decelerating gradually for 2.5 laps as a final ‘warm down’. All samples were taken before and after an early morning hard work session and prior to provision of feed or supplement. The light training sessions involved approximately 2.5 ‘warm-up’ laps of the track at a moderate pace and then a light jog at around 19 km/h for 9-12 km. To provide consistency in training tempo and demand between horses and between sampling events, the same driver was used for all horses in all sessions. The various aspects of the training, including duration, session times, distances, speeds and heart rate parameters were monitored regularly in the first cohort to assess consistency between horses and between pre- and post-supplementation stages project.

Each horse had replicate samples taken for assessment of pre-exercise blood and plasma as well as post-exercise blood, plasma and sweat at both baseline and post-supplementation stages. The replicate samples for each phase of testing were averaged to represent a single representative blood, plasma or sweat sample for each animal at both the baseline and subsequent post-supplementation stages. The data sets of both cohorts were analysed separately to assess the consistency of amino acid composition in sweat and responses to amino acid supplementation in two different cohorts of animals. Before commencement of the supplementation baseline levels of plasma amino acids for each horse were established by taking blood samples before and immediately following the exercise training regimes on four or five separate occasions. After 35 days of supplementation in the first cohort, a second set of samples was taken on three separate occasions whilst the horses remained on the supplement to establish the post-supplementation compositions of the plasma and sweat. The same approach was taken for the second cohort, where baseline levels were established for each horse during four separate sampling events prior to supplementation. After 40 days of supplementation for the second cohort, a second set of samples were taken on four separate occasions whilst the horses remained on the supplement to establish the post-supplementation compositions of the plasma and sweat.

Blood was collected from the jugular vein of each horse in a 20 mL syringe and transferred directly to a labelled 9 mL sodium heparin Vacutainer®. Sweat was collected on completion of each horse's training session by scraping a sterile 70 mL sample jar in an upward motion over the surface of the horse's coat to allow the fluid to run into the container. In the first cohort, a combined sweat sample was collected from three areas of the body: the chest between and immediately above the forelegs; the sides and underbelly of the torso; and the insides of the upper portion of the hind legs. In the second cohort, sweat was collected separately from the same three regions to determine whether any differences in sweat composition of amino acids occurred at different locations on the body. Once collected, the each sweat sample was transferred to a sterile Monovette® tube (Sarstedt Australia Pty Ltd) and stored immediately following collection in a chilled container for transport to the laboratory. All blood samples were collected prior to supplementation and feeding.

Following the blood draw, a 100 μL sample of whole blood was drawn into heparinised capillary tube (Bacto Laboratories, Liverpool, NSW). The sample was immediately expelled from the capillary tubes into the sample well of an iSTAT CGs8 cartridge. All air bubbles were removed from the samples prior to the cartridges being closed. The cartridge was analysed by iSTAT clinical analyser for measures of haematocrit (Hct), haemoglobin (Hb), ionized calcium (iCa), glucose (Glu), sodium (Na), potassium (K), pH, bicarbonate (HCO₃), blood gases (pO₂, pCO₂, TCO₂, sO₂) and base excess. Prior to each testing session, the i-STAT analyser was calibrated according to manufacturer's specifications by an electronic stimulation and Level 2 i-STAT control solution (i-STAT Corporation, New Jersey, USA). Cartridges were stored prior to use as per manufacturers instructions (2-8° C.), and were removed to room temperature approximately 5 min prior to use.

The plasma fraction was isolated from the blood samples via centrifugation (3000 rpm, 10 min) and the plasma supernatant was subsequently transferred to sterile 2 mL Eppendorf tubes. Aliquots of sweat samples were removed from the Monovette® tubes and centrifuged at 2000 rpm for five minutes. The clear supernatant was transferred to a clean tube for extraction. The amino acid composition of samples was determined via EZ:Faast™ derivatisation (Phenomenex Inc.) followed by GC/FID analysis. The EZ:Faast™ procedure consists of a solid phase extraction step, followed by derivatisation and a liquid/liquid extraction. All samples were derivatised according to the manufacturer's protocol, with the following modifications: i) addition of 200 μL of sterile de-ionised water to the initial reaction mixture for all plasma samples; ii) addition of 200 μL 0.1 M HCl to the initial reaction mixture for all sweat samples. Analysis of the EZ:Faast™ derivatised samples was performed on a Hewlett Packard HP 6890 series GC system fitted with a flame ionisation detector and ZB-PAAC-MS column (10 m×0.25 mm i.d.), supplied by Phenomenex Inc. The instrument method comprised split injection (ratio 15:1) with injector temperature 250° C. and column flow rate 0.5 ml/min. Injection volume was set at 2.5 μL for all samples. The oven programme comprised an initial temperature of 110° C., increasing 32° C./min to 320° C. (run time=8.56 min). Target compounds were identified according to pre-established retention times of analytical standards, with quantification calibrated against the signal response of an internal standard.

### Statistical Analysis

Comparisons of blood parameters, exercise regimes and amino acid concentrations between sample sets were completed by one-way ANOVA using Statistica™ 12 (Statsoft, Tulsa, Okla., USA). Effect sizes were calculated and interpreted accordingly to assess the magnitude of difference between the means for haematocrit and haemoglobin assessed at baseline and after supplementation (Cohen's d; small=0.2-0.49, moderate=0.5-0.79, large≥0.8). Samples from both cohorts were pooled for comparison of pre- and post-supplementation levels of total amino acids in resting plasma using paired-samples t-test in Statistica. Correlation analyses were performed on the plasma resting plasma levels of amino acids and the corresponding haematocrit and haemoglobin levels in the horses at baseline and again following supplementation period using Statistica. Levels of statistical significance were set at p<0.05.

### Results

The training regime was kept as consistent as possible throughout the experimental periods to enable meaningful comparisons to be made between the various measures taken before and after supplementation. A range of parameters from the training regime were objectively assessed in order to identify potential variations in exercise load between the two stages for the first cohort. The data are summarised in Table 5. It was apparent that 75% of the measured parameters did not show significant differences between the two stages of assessment. It was expected that the training mean speeds and session distances would vary to some degree based upon the prevailing season, weather and track conditions. In light of this, it was concluded that the training regimes were sufficiently consistent between the pre- and post-supplementation arms of the study to allow comparisons to be made. A similar regime was applied to the second cohort.

The sweat collected from the three collection sites on each animal in cohort 1 was pooled for each horse and displayed high variability for the amino acid concentrations (Table 6). The sweat collected from each sample site in the cohort 2 samples were analysed separately revealing that the chest samples had a total amino acid concentration of 3,214±411 μmol/L compared with the underbelly at 2,777±428 μmol/L and the hind legs at 1,876±315 μmol/L (P<0.05). The chest area sweat also had the highest levels of serine, histidine and threonine

TABLE 5
Comparisons of baseline and post-supplement training
parameters for the First Cohort of horses.

| Parameter | Pre-supplement | Post-supplement |
|---|---|---|
| Training duration (s) | 301 ± 13 | 259 ± 43 |
| Training mean heart rate (bpm) | 208 ± 8 | 214 ± 6 |
| Training peak heart rate (bpm) | 224 ± 6 | 230 ± 4 |
| Training peak speed (km/h) | 51 ± 0.6 | 50 ± 2 |
| Session time (s) | 1049 ± 24 | 1062 ± 145 |
| Session mean heart rate (bpm) | 158 ± 3 | 156 ± 9 |
| Session peak heart rate (bpm) | 224 ± 6 | 229 ± 5 |
| Session peak speed (km/h) | 54 ± 0.9 | 53 ± 2 |
| Training distance (km) | 3.8 ± 0.2 | 3.1 ± 0.5* |
| Training mean speed (km/h) | 44 ± 1 | 41 ± 2* |
| Session mean speed (km/h) | 27 ± 0.5 | 22 ± 1* |
| Session distance (km) | 7.0 ± 0.3 | 6.3 ± 0.6* |

Values are means ± SE, n = 5.
Significant difference from values obtained at baseline at *P < 0.05.

and was deemed the easiest and safest collection site for the animal handlers. This sampling site was therefore used for the reporting of sweat data for cohort 2. At baseline, the mean loss in body weight which occurred during exercise for cohort 2 was 5.1±0.7 kg.

Twenty-two amino acids were detected and quantified in post-exercise plasma taken from the horses. Comparisons between the plasma compositions from cohort 1 and cohort 2 revealed that the average plasma profile patterns were similar across the two studies (Table 6), with glycine as the major plasma amino acid, followed by alanine, glutamine, valine and serine, comprising 61-64% of the plasma amino acids. The total levels of amino acids in the plasma from the two cohorts were similar in both individual amino acid magnitude and distribution.

The amino acid compositions of the sweat were significantly different from those of the corresponding plasma samples for both cohorts (Table 6). The average total concentrations of amino acids in the sweat samples were double that of the plasma for cohort 1 (P<0.05) and although 1.2 times higher in cohort 2, but this latter difference did not reach levels of statistical significance. The sweat contained five amino acids which were consistently present in higher concentrations in the sweat compared with the corresponding plasma levels for both study cohorts and included serine (3.9-5.4 times higher), glutamic acid (7.0-9.5 times higher) histidine (4.3-4.5 times higher), phenylalanine (1.9-3.4 times higher), and aspartic acid.

Aspartic acid was not detected in the plasma from the horses in cohort 1 but was present at 262±29 μmol/L in the sweat. Similarly, it was measured at 2±2 μmol/L in plasma from cohort 2 compared with a corresponding 154±21 μmol/L in the sweat. Alanine, leucine, valine, proline and tyrosine were higher in sweat relative to plasma in cohort 1 but not cohort 2. Both valine and ornithine were more concentrated in the sweat relative to the plasma in cohort 1 but were less concentrated in the sweat in cohort 2. Glutamine, cystine, methionine and asparagine were consistently lower in concentration in the sweat relative to the plasma in both cohorts. Glycine and tryptophan were lower in the sweat compared with plasma in cohort 2 and present at equivalent levels in both matrices in cohort 1.

TABLE 6
The mean concentrations of amino acids in post-exercise sweat and plasma
samples taken from horses prior to commencement of supplementation

| Amino acid type based | Post- | | | | |
|---|---|---|---|---|---|
| on sweat levels higher | exercise | | | | |
| than corresponding | amino | Cohort 1 | | Cohort 2 | |
| plasma levels | acids | Sweat | Blood plasma | Sweat | Blood plasma |
| Type A: Sweat amino | Serine | 893 ± 103 | 164 ± 8* | 791 ± 108 | 202 ± 14* |
| acid concentrations | Glutamic | 429 ± 53 | 45 ± 7* | 167 ± 25 | 24 ± 2* |
| higher than or equivalent | acid | | | | |
| to plasma levels | Histidine | 396 ± 184 | 88 ± 5* | 206 ± 28 | 48 ± 6* |
| | Aspartic | 262 ± 29 | 0.0* | 154 ± 21 | 2 ± 2* |
| | acid | | | | |
| | Phenylalanine | 222 ± 48 | 66 ± 2* | 131 ± 18 | 69 ± 4* |
| Type B: Amino acids | Alanine | 594 ± 69 | 392 ± 12* | 489 ± 68 | 345 ± 12 |
| with variable sweat | Leucine | 489 ± 106 | 144 ± 2* | 128 ± 20 | 138 ± 7 |
| concentrations relative to | Lysine | 448 ± 136 | 156 ± 7 | 150 ± 23 | 96 ± 3* |
| the plasma | Valine | 389 ± 49 | 266 ± 6* | 137 ± 27 | 207 ± 14* |
| concentrations, | Proline | 164 ± 18 | 96 ± 3* | 111 ± 22 | 110 ± 4 |
| depending on study | Tyrosine | 158 ± 31 | 66 ± 2* | 87 ± 15 | 77 ± 4 |
| cohort | Ornithine | 129 ± 35 | 83 ± 2 | 39 ± 5 | 59 ± 4*^{‡} |
| Type C: Sweat amino | Glycine | 616 ± 65 | 607 ± 34 | 409 ± 55 | 637 ± 30* |
| acid concentrations lower | Tryptophan | 30 ± 8 | 44 ± 1 | 13 ± 2 | 60 ± 7* |
| than or equivalent to | Cystine | 21 ± 3 | 36 ± 2* | 3 ± 1 | 28 ± 2* |
| plasma levels | Methionine | 18 ± 3 | 29 ± 2* | 7 ± 2 | 22 ± 1* |
| | Cystathionine | 15 ± 5 | 14 ± 6 | 0.2 ± 0.2 | 9 ± 4 |
| | Glutamine | 14 ± 9 | 286 ± 15* | 9 ± 5 | 251 ± 19* |
| | Hydroxylysine | 2 ± 2 | 30 ± 3* | 3 ± 2 | 0 ± 0 |
| | Asparagine | 1 ± 1 | 30 ± 2* | 8 ± 3 | 21 ± 1* |
| | Total^{#} | 5,696 ± 932 | 2,834 ± 18* | 3,213 ± 411 | 2,584 ± 101 |

Values are means ± SE (μmol/L).
Sample size: cohort 1 n = 5; cohort 2 n = 6.
*Plasma values significantly different compared with the corresponding sweat values (P < 0.05).
^{#}Total includes some minor amino acid derivatives not shown: α-aminopimelic acid, α-aminoadipic acid, glycine-proline and β-aminoisobutyric acid.

### Changes in Resting Blood, Plasma and Sweat Post-Supplementation

Average amino acid levels were assessed in resting plasma samples at baseline and after supplementation to assess potential changes in metabolic homeostasis following amino acid supplementation (Table 7). Cohort 1 had a higher average baseline level of total plasma amino acids at 2,293±68 μmol/L compared with cohort 2 at 2,044±135 μmol/L, but the resting levels displayed similar profile characteristics between the two years. Following supplementation, average total plasma amino acid levels increased in both cohorts compared with their corresponding mean baseline levels to 2,674±41 μmol/L and 2,663±124 μmol/L respectively (P<0.05). The average total levels of amino acids in plasma were therefore equivalent for the two cohorts post-supplementation. The two cohorts were pooled to perform a paired-samples t-test to compare plasma amino acid concentrations at baseline and post-supplement with a Bonferroni correction. There were significant increases observed in the resting plasma after the supplementation period compared with the baseline levels for glycine (baseline: 582 μmol/L vs post-supplement: 769 μmol/L), threonine (93 μmol/L vs 118 μmol/L), serine (175 μmol/L vs 312 μmol/L), and glutamine (213 μmol/L vs 343 μmol/L) (n=9, P<0.002). The average total amino acid concentration in plasma after supplementation (mean=2,669 μmol/L, SD=165) was also assessed using a paired-samples t-test and was found to be significantly higher compared with the levels observed prior to supplementation (mean=2,138 μmol/L SD=313) (t(8)=−4.29, P<0.003). The data indicated that the process of amino acid supplementation immediately after exercise resulted in increased circulatory levels of amino acid levels in resting plasma.

As well as having a lower plasma total amino acid content at baseline, cohort 2 also had a lower initial average total amino acid level in the sweat at 3,213±411 μmol/L compared with the cohort 1 level of 5,696±932 μmol/L (Table 8). Following supplementation, the average total sweat amino acid levels did not increase significantly for cohort 1 at 6,228±546 μmol/L, but more than doubled in concentration for cohort 2 at 8,682±563 μmol/L (P<0.05). All of the amino acids measured were observed at higher levels in the sweat post-supplement compared with baseline levels for cohort 2 (P<0.05).

TABLE 7
Comparisons of the amino acid composition of pre-exercise plasma
before and after supplementation for both cohort 1 and cohort 2.

| | Cohort 1 | | Cohort 2 | | | | |
|---|---|---|---|---|---|---|---|
| | Plasma concentrations | | Plasma concentrations | | Horses not in | | |
| | (Mean ± SE) | | (Mean + SE) | | work | Literature values^{a} | |
| | Baseline | Post-supplement | Baseline | Post-supplement | (Mean ± SE) | Mean (range) | |
| Amino acid | (n = 5) | (n = 5) | (n = 6) | (n = 4) | (n = 7) | (n = 10) | |
| Glycine | 588 ± 42 | 795 ± 29* | 608 ± 49 | 736 ± 29 | 423 ± 57 | 487 | (298-641) |
| Serine | 167 ± 5 | 338 ± 15* | 198 ± 26 | 279 ± 24 | 287 ± 34 | 223 | (88-332) |
| Glutamine | 256 ± 18 | 354 ± 16* | 172 ± 14 | 330 ± 17* | 393 ± 24 | 322 | (179-440) |
| Valine | 228 ± 7 | 191 ± 4* | 169 ± 5 | 177 ± 7 | 291 ± 21 | 301 | (199-457) |
| Alanine | 134 ± 9 | 189 ± 29 | 127 ± 9 | 185 ± 14* | 207 ± 26 | 245 | (131-420) |
| Proline | 81 ± 4 | 90 ± 4 | 103 ± 9 | 110 ± 8 | 121 ± 15 | NR | |
| Leucine | 98 ± 5 | 90 ± 4 | 96 ± 5 | 106 ± 3 | 157 ± 16 | 144 | (73-252) |
| Threonine | 96 ± 8 | 115 ± 5 | 89 ± 5 | 121 ± 5* | 184 ± 22 | 171 | (73-235) |
| Tryptophan | 51 ± 3 | 50 ± 2 | 67 ± 8 | 67 ± 3 | 65 ± 8 | NR | |
| Lysine | 116 ± 7 | 75 ± 6* | 65 ± 6 | 87 ± 5* | 156 ± 27 | 144 | (64-201) |
| Tyrosine | 58 ± 2 | 53 ± 1* | 64 ± 6 | 67 ± 4 | 90 ± 10 | 93 | (48-100) |
| Phenylalanine | 56 ± 3 | 48 ± 3 | 59 ± 4 | 60 ± 2 | 76 ± 8 | 80 | (50-95) |
| Isoleucine | 53 ± 1 | 58 ± 3 | 52 ± 3 | 53 ± 4 | 83 ± 7 | 78 | (66-130) |
| Ornithine | 74 ± 3 | 58 ± 3* | 51 ± 5 | 68 ± 2* | 77 ± 10 | 56 | (24-81) |
| Histidine | 90 ± 3 | 54 ± 4* | 33 ± 5 | 61 ± 4* | 112 ± 16 | 76 | (60-97) |
| Cystine | 40 ± 3 | 23 ± 2* | 27 ± 3 | 31 ± 2 | 50 ± 4 | 28 | (10-51) |
| Methionine | 25 ± 3 | 22 ± 2 | 21 ± 2 | 19 ± 0.3 | 38 ± 4 | 33 | (23-45) |
| Asparagine | 24 ± 3 | 32 ± 1* | 21 ± 3 | 32 ± 3* | 8 ± 1 | NR | |
| Glutamic acid | 17 ± 5 | 31 ± 3 | 11 ± 1 | 21 ± 1* | 26 ± 3 | 19 | (<10-32) |
| Cystathionine | 5 ± 1 | 4 ± 2 | 1 ± 0.4 | 1 ± 0.7 | 50 ± 4 | NR | |
| Aspartic acid | 0.0 | 0 | 0 ± 0 | 14 ± 2* | 8 ± 1 | <10 | (7-11) |

| | | | | | | |
|---|---|---|---|---|---|---|
| Total | 2,293 ± 68 | 2,674 ± 41* | 2,044 ± 135 | 2,663 ± 124* | 3,450 ± 61 | 2,754^{b} |

Units are μmol/L.
*Significant differences between baseline and post-supplement concentrations within each cohort.
^{a}Results reported for non-grazing mixed breed horses, 2-20 years of age (McGorum and Kirk, 2001); NR indicates not reported.
^{b}There were four amino acids reported in the current study which were not reported in the McGorum and Kirk (2001) study. Thus the values for proline tryptophan, asparagine and cystathione from the current study for the horses out of work were substituted into the calculation of totals for the purposes of reference comparison. The reference data reported additional values for arginine, taurine and citrulline which were not measured by the current technique (representing an additional combined 195 μmol/L).

TABLE 8
Comparisons of the amino acid composition of baseline
and post-supplement sweat for cohort 1 and cohort 2.

| | Cohort 1 | | Cohort 2 | |
|---|---|---|---|---|
| | Sweat concentrations | | Sweat concentrations | |
| | Baseline | Post-supplement | Baseline | Post-supplement |
| Amino acid | (n = 5) | (n = 5) | (n = 6) | (n = 4) |
| Serine | 893 ± 103 | 1,588 ± 178* | 791 ± 108 | 1,752 ± 91* |
| Glycine | 616 ± 65 | 787 ± 61 | 409 ± 55 | 1,119 ± 111* |
| Alanine | 594 ± 69 | 792 ± 87 | 489 ± 68 | 1,292 ± 78* |
| Leucine | 489 ± 106 | 166 ± 11* | 128 ± 20 | 485 ± 64* |
| Lysine | 448 ± 136 | 291 ± 15 | 150 ± 23 | 473 ± 52* |
| Glutamic Acid | 429 ± 53 | 411 ± 31 | 167 ± 25 | 571 ± 106* |
| Valine | 389 ± 49 | 154 ± 14* | 137 ± 27 | 325 ± 33* |
| Histidine | 396 ± 184 | 924 ± 175 | 206 ± 28 | 600 ± 206* |
| Aspartic Acid | 262 ± 29 | 221 ± 21 | 154 ± 21 | 372 ± 32* |
| Phenylalanine | 222 ± 48 | 152 ± 8 | 131 ± 18 | 304 ± 32* |
| Isoleucine | 210 ± 36 | 87 ± 7* | 66 ± 10 | 218 ± 28* |
| Proline | 164 ± 18 | 153 ± 9 | 111 ± 22 | 206 ± 19* |
| Threonine | 165 ± 20 | 189 ± 22 | 97 ± 16 | 302 ± 19* |
| Tyrosine | 158 ± 31 | 101 ± 5 | 87 ± 15 | 202 ± 23* |
| Ornithine | 129 ± 35 | 85 ± 10 | 39 ± 5 | 82 ± 13* |
| Tryptophan | 30 ± 8 | 19 ± 1 | 13 ± 2 | 33 ± 2* |
| Cystine | 21 ± 3 | 5 ± 2* | 3 ± 1 | 13 ± 1* |
| Methionine | 18 ± 3 | 8 ± 3* | 7 ± 2 | 30 ± 11* |
| Cystathionine | 15 ± 5 | 3 ± 2 | 0.2 ± 0.2 | 4 ± 2* |
| Glutamine | 14 ± 9 | 38 ± 4* | 9 ± 5 | 134 ± 32* |
| Asparagine | 1 ± 1 | 18 ± 14 | 8 ± 3 | 58 ± 21* |
| Total^{‡} | 5,696 ± 932 | 6,228 ± 546 | 3,213 ± 411 | 8,682 ± 563* |

Values are means ± SE (μmol/L).
Significant difference from values obtained pre-supplement at *P < 0.05.
^{‡}Total includes some minor amino acid derivatives not shown in the above table: α-aminopimelic acid, α-aminoadipic acid, glycine-proline, and β-aminoisobutyric acid.

The mean resting haematocrit and haemoglobin levels were 0.41±0.025 and 141+8.6 g/L respectively for the four cohort 2 horses at baseline who completed the supplementation program. Following supplementation, large increases (Cohen's d>0.8) were observed for both parameters where the resting haematocrit increased to 0.46±0.015 and haemoglobin increased to 155±5.3 g/L. The correlation analyses of the resting levels of plasma amino acids indicated that threonine was the only amino acid with a strong correlation to the resting blood levels of haemoglobin. After the supplementation period, a number of amino acids showed strong associations with haemoglobin where histidine, valine, asparagine, glutamic acid, glutamine and lysine displayed R²>0.98. In an additional horse with initial low haemoglobin (117 g/L), the animal was given 30 g per day of amino acid supplement formulated to contain only the 14 amino acids identified as representing the major amino acids components lost in sweat (serine, glutamic acid, histidine, leucine, lysine, aspartic acid, alanine, glycine, phenylalanine, valine, isoleucine, proline, threonine, and tyrosine). The proportions of amino acids were adjusted to reflect the relative losses observed for the amino acids in the sweat for these animals. These amino acids were pre-mixed in the appropriate proportions and were combined with water 3:1 with xanthan gum added to assist forming a paste delivery system via 60 mL syringes, which was well received by the animal. The paste comprised 30 g amino acids with 500 mg glucose per serve. Haemoglobin increased to 125 g/L after four days of supplementation. Haemoglobin increased further to 132 g/L after 13 days of supplementation. In the 13 day period, the red cell count was also increased from 6.9×10′²/L to 7.7×10¹²/L. These data support that the amino acid supplementation can result in elevated haemoglobin levels, particularly where the starting level is below 140 g/L.

The performance assessments from the first cohort revealed that average recovery heart rate, measured 10 minutes after completing exercise, was reduced from 83.3±5.6 pre-supplement to 77.2±4.3 bpm (P<0.05) following the supplementation period. The conditions of the animals were monitored throughout the experimental period and the trainer provided valuable feedback on each of the animals for both study cohorts, which is summarised in Table 9. The major features subjectively assessed and reported by the horse trainer were that the supplementation resulted in a range of improvements including health, well-being and performance. Of specific note were comments referring to improved coats, bright eyes as well as better maintenance of racing condition and development of muscle mass during the testing period.

Analyses for the two cohorts revealed that the sweat facilitated losses of amino acids (SFLAA) could be substantial during a training or exercise regime with horses losing an estimated 1.6-3 g of free amino acids. This loss would result in an immediate increase in the demand for the utilisation of the muscle reserves of amino acids via a catabolic response. To set this in context, although the plasma volume in horses is variable between breeds and the levels of fitness and hydration, an average 450 kg horse would have a plasma volume of around 16 L with a corresponding red cell volume around 20 L. The total amino acid loading in the 16 L plasma volume was thus calculated as 3.8-4.3 g across the two cohorts of horses in the present study. Based upon the above calculations, approximately 40-70% of the plasma loading of amino acids may be lost through sweat during exercise that resulted in a 1% reduction of the body mass via sweating. This indicated that sweating increased the requirement for amino acids to enter circulation via the catabolic turnover of muscle storage proteins. It was thus argued that the loss of amino acids via sweat would place additional demands on muscle reserves to replenish plasma levels required to support metabolic processes including energy production (glucose-alanine cycle), oxygen delivery, muscle repair and recovery.

TABLE 9
Comments from the trainer following the First Cohort and
the Second Cohort supplementation periods.

| First Cohort | Second Cohort |
|---|---|
| Horse D: | Horse A: |
| “Brighter” and “more active” following | Saw a significant increase in rear muscle bulk. |
| supplementation. | Supplement improved health, well-being and |
| Horse R: | performance. |
| “Brighter” and “more active” following | Physical signs: bright, healthy gums, brilliant |
| supplementation. | coat. |
| Last run described as “pretty good”. | Performance: times improved |
| | Previously unable to maintain weight but was |
| | able to whilst using supplement. |
| Horse F: | Horse B: |
| “Brighter” and “more active” following | Saw a significant increase in rear muscle bulk. |
| supplementation. | Healthy but suffered from fetlock problems |
| Saw a reduction in body fat and increased | (present both pre- and post-supplement). |
| muscle mass. | Supplement improved health, well-being and |
| Appearance became leaner and amore | performance. |
| athletic. | Physical signs: eyes bright, healthy gums, |
| | brightness of coat. |
| | Performance: times improved |
| Horse H: | Horse M: |
| Horse was significantly “brighter” | Saw an increase in rear muscle bulk. |
| following supplementation, for example | Supplement improved health, well-being and |
| “pig-rooting” after runs. | performance. |
| Gained 20 kgs in 14 days (younger horse, | Physical signs: significant improvement in |
| still growing) | brightness of eyes, gums, coat. Significant |
| Note: Although the body condition scores did not | improvement - very alert, “fruity”. |
| improve, the trainer believed that this was due to the | Performance: Originally a “non-trier”, saw |
| fact that this younger horse was still growing. | significant improvements with supplement, |
| | times improved 3-4 seconds, sustained speed |
| | for longer. |
| Horse N: | Horse C: |
| Initial condition of horse - poor condition | Saw an increase in rear muscle bulk. |
| despite no known illness being detected. | Health: Currently healthy but previous broken |
| CPK always elevated. After | pelvis. |
| supplementation CPK levels normalised. | Supplement improved health and well-being. Did |
| Much “Brighter” and more active | not improve performance, attributed to previous |
| following supplementation. | injury. |
| “Eating better”. | Physical signs: improvements seen in gums, |
| Following supplementation, an experienced | coat, brightness. |
| driver commented that the horse “looks | Normally during racing loses condition. |
| enormous”. This is not a comment that had | During supplementation did not lose |
| ever been made before in regards to this | condition. |
| particular horse. | With supplementation maintained weight |
| | and condition. Had not previously |
| | maintained weight and condition. |

This loss of 1.6-3 g of free amino acids would result in an immediate increase in the demand for the utilisation of the muscle reserves of amino acids via a catabolic response. Proteolysis would allow for the replacement of the amino acids required for the supportive metabolic processes including energy production (glucose-alanine cycle), oxygen delivery and, and muscle repair recovery. Comparisons of the amino acid profiles in sweat with the corresponding levels in the plasma revealed a group of 6 amino acids, referred to as Type A, which were present in the sweat at substantially higher concentrations compared to the corresponding levels in plasma in both studies and the Type B amino acids which were significantly higher in sweat compared with plasma in one of the studies. Some of these amino acids were non-essential amino acids and can be synthesised by the horse, but under conditions of prolonged exercise or exposure to heat, these metabolites may become conditionally essential if synthesis cannot meet demand. Serine was the major amino acid measured in the sweat. In addition to its requirements for protein synthesis, serine and its derivatives form functional groups in key membrane phospholipids such as phosphatidylserine, phosphotydylcholine and phosphotidylethanolamine. Serine is the immediate precursor for the synthesis of glycine which is the most abundant amino acid in the horse plasma. The formation of glycine from serine also generates methylene-tetrahydrofolate from tetrahydrofolate which is essential for the synthesis of nucleic acids. Thus substantial sweat facilitated losses of serine could lead to a broad range of metabolic deficits with impact on performance and recovery if body supply could not keep up with demand.

The supplementation used in the first cohort utilized a commercial amino acid supplement designed for addressing fatigue in humans (Dunstan 2013, 2014), whereas the second cohort tested a supplement that was designed to specifically address losses via sweating in horses by providing the amino acids identified as Type A amino acids. The formulation included 6 amino acids with the highest concentrations in sweat relative to plasma: serine, lysine, histidine, leucine, glutamic acid and aspartic acid. As the major components lost in the horse sweat, these amino acids represented 60% of the amino acids in the formulation together with alanine, glycine, phenylalanine, valine, isoleucine, proline, threonine and tyrosine making up the remaining 40%. Valine was included as a potential high loss essential amino acid and glycine was included because although its concentration in the sweat was equivalent to that in the plasma, it represented the second or third most abundant amino acid lost in sweat.

The key result of amino acid supplementation over 40 days was that both products resulted in elevating the resting plasma levels to equivalent levels of 2,674±41 μmol L⁻¹ and 2,663±124 μmol L⁻¹ respectively. This result demonstrated that the supplementation process was effective in altering amino acid homeostasis in the horses in both cohorts. Without wishing to be bound by theory, the elevation of average total amino acid concentrations to similar levels suggests a hypothesis whereby there exists a plasma optimised level of amino acids (POLAA) which may not be further increased by supplementation under conditions of training and regular work. The data from the horses that had been rested from work revealed that the total amino acid concentration in plasma increased to a level approximately 30% higher than the POLAA assessed post supplementation in the working horses. This was interpreted to reflect that under conditions of regular intense training and racing, the animals operated on a different homeostasis with lower levels of circulating plasma amino acids. Under high intensity training conditions, the continued daily cycles of exercise require activation of the catabolic response to provide amino acids from the muscle stores. Following the exercise, the horses have a limited time for replenishment and recovery which could place excessive demand on the delivery of amino acids for whole body metabolism. Following a period of resting, the stores become replenished and the homeostasis shifts to higher level of plasma amino acid concentrations as observed in the horses out of work. Without wishing to be bound by theory, the inventors propose that prolonged catabolism stimulated by either over training or infectious challenge could lead to diminished amino acid stores where the body cannot meet demand via de novo synthesis. Also without wishing to be bound by theory, the inventors propose that a simple blood plasma test for total levels of amino acids would provide an indication of amino acid status in horses to determine supplementation requirements to optimise performance and condition during periods of training and racing. This would provide a tool for managing and optimising the dosage levels throughout training periods.

The results described here indicated that a simplified formulation of 14 amino acids was as effective in achieving POLAA as the more complex Fatigue Reviva™ formulation which contained 20 amino acids, fructo-oligosaccharide (FOS), malic acid, citric acid, succinic acid, ribose, 13 minerals and 13 vitamins. The supplement used in the second cohort resulted in the horses having higher levels of the key amino acids included in the formulation, including proline, leucine, tryptophan, tyrosine, phenylalanine, cystine and aspartic acid which would have potential benefit for the animals' wellbeing.

The corresponding levels of amino acids in sweat also increased substantially following supplementation. This was more striking in the second cohort which increased from 3,213±411 μmol L⁻¹ to 8,682±563 μmol L⁻¹ after supplementation (Table 8). The higher levels of amino acids in sweat were largely attributable to differences in serine, glycine, alanine, leucine, lysine and glutamic acid, which were the major components of the second supplement. These results were interpreted as indicating that the supplement formulated on the basis of sweat facilitated losses was extremely efficient at delivering the amino acids. The increases in amino acid composition in the sweat following supplementation also suggest that amino acids measured in the sweat collected from the horses reflect metabolic homeostasis and the nutritional status of the horses.

The feedback from the trainer from both cohorts provided vital subjective evidence that the supplementation provided benefits in regard to the general condition of the horses, supporting development of muscle mass, glossy coats, bright eyes and a capacity to work hard for longer. Without wishing to be bound by theory, it is suggested that the higher levels of plasma amino acids would potentially be able to better support provision of energy during work and protein synthesis supporting processes of oxygen delivery, muscle growth and recovery from exercise. This is evidenced by the results in Table 9 indicating maintenance of body weight during the supplementation period with two of the horses showing increases in haemoglobin levels after the supplementation period.

### REFERENCES

Dunstan, R. H., Sparkes, D. L., Roberts, T. K., Crompton, M. J., Gottfries, J., & Dascombe, B. J. (2013). Development of a complex amino acid supplement, Fatigue Reviva™, for oral ingestion: initial evaluations of product concept and impact on symptoms of sub-health in a group of males. Nutrition journal, 12(1), 115.
Dunstan, R. H., Sparkes, D. L., Roberts, T. K., & Dascombe, B. J. (2014). Preliminary evaluations of a complex amino acid supplement, fatigue Reviva™, to reduce fatigue in a group of professional male athletes and a group of males recruited from the general public. FNS, 5, 231-235.
Evans, C. R. Hugh Dunstan, Tony Rothkirch, Tim K. Robertsl, Karl L. Reichelt, Robyn Cosford, Gary Deed, Libby B. Ellis, Diane L. Sparkes. (2008) Altered amino acid excretion in children with autism, Nutritional Neuroscience 11 (1), 9-17. IF=1.3
Macintosh, B. R., & Rassier, D. E. (2002). What is fatigue? Canadian Journal of Applied Physiology, 27(1), 42-55.
McDonald, R. E., Fleming, R. I., Beeley, J. G., Bovell, D. L., Lu, J. R., Zhao, X., . . . Kennedy, M. W. (2009). Latherin: A surfactant protein of horse sweat and saliva. PLOS One, 4(5), e5726.
Niblett, S. N. (2007). Hematologic and urinary excretion anomalies in patients with chronic fatigue syndrome. Exp Biol Med, 232, 1041-1049.

1. An amino acid composition comprising histidine, serine and lysine, wherein the histidine, serine and lysine together comprise at least about 25% of the total weight of amino acids in the composition.

2. The amino acid composition of claim 1, wherein the histidine, serine and lysine comprise at least about 30% of the total weight of amino acids in the composition.

3. The amino acid composition of claim 1 or 2, wherein the histidine, serine and lysine comprise between about 30% and 50% of the total weight of amino acids in the composition.

4. The amino acid composition of claim 3, wherein the histidine, serine and lysine comprise between about 32% and 47% of the total weight of amino acids in the composition.

5. The amino acid composition of any one of claims 1 to 4, wherein the histidine comprises between about 10% and 21%, the serine comprises between about 13% to 16%, and the lysine comprises between about 9% and 10% of the total weight of amino acids in the composition.

6. The amino acid composition of any one of claims 1 to 5, further comprising at least one of ornithine and glycine.

7. The amino acid composition of claim 6, wherein, where present, the ornithine comprises at least about 12%, and the glycine comprises at least about 8%, of the total weight of amino acids in the composition.

8. The amino acid composition of claim 6 or 7, wherein the composition comprises histidine, serine, lysine, ornithine and glycine, and wherein these amino acids comprise at least about 40% of the total weight of amino acids in the composition.

9. The amino acid composition of claim 6 or 7, wherein the composition comprises histidine, serine, lysine, ornithine and glycine, and wherein these amino acids comprise between about 50% and 80% of the total weight of amino acids in the composition.

10. The amino acid composition of any one of claims 1 to 5, further comprising at least one of glutamine, glutamic acid, leucine and aspartic acid.

11. The amino acid composition of claim 10, wherein, where present, the glutamine and/or glutamic acid comprises at least about 10%, the leucine comprises at least about 10%, and the aspartic acid comprises at least about 7% of the total weight of amino acids in the composition.

12. The amino acid composition of claim 10 or 11, wherein the composition comprises histidine, serine, lysine, glutamine and/or glutamic acid, leucine and aspartic acid, and wherein these amino acids comprise at least about 35% of the total weight of amino acids in the composition.

13. The amino acid composition of claim 10 or 11, wherein the histidine, serine, lysine, glutamine and/or glutamic acid, leucine and aspartic acid comprise between about 40% and 60% of the total weight of amino acids in the composition.

14. An amino acid composition comprising histidine, serine, ornithine and glycine, wherein the histidine, serine, ornithine and glycine together comprise at least about 30% of the total weight of amino acids in the composition.

15. The amino acid composition of claim 14, wherein the histidine, serine, ornithine and glycine together comprise at least about 60% of the total weight of amino acids in the composition.

16. The amino acid composition of claim 14, wherein the histidine, serine, ornithine and glycine together comprise at least about 64% of the total weight of amino acids in the composition.

17. The amino acid composition of claim 14, wherein the histidine, serine, ornithine and glycine together comprise at least about 76% of the total weight of amino acids in the composition.

18. An amino acid composition comprising serine, alanine, glycine, histidine and proline, wherein the serine, alanine, glycine, histidine and proline together comprise at least about 20% of the total weight of amino acids in the composition.

19. An amino acid composition comprising serine, glutamic acid, histidine, leucine, lysine, aspartic acid, alanine, glycine, phenylalanine, valine, isoleucine, proline, threonine, and tyrosine.

20. The amino acid composition of any one of claims 1 to 19, wherein the composition is a dietary supplement.

21. The amino acid composition of any one of claims 1 to 20, wherein the composition promotes or assists recovery from exercise, illness or injury in a subject.

22. The amino acid composition of any one of claims 1 to 20, wherein the composition assists survival in hot climates.

23. The amino acid composition of any one of claims 1 to 20, wherein the composition promotes or assists exercise performance.

24. A method for promoting or assisting recovery from exercise, illness or injury in a subject, the method comprising administering to the subject an effective amount of an amino acid composition of any one of claims 1 to 20.

25. The method of claim 24, wherein the illness is chronic fatigue.

26. A method for assisting survival of a subject in a hot climate, the method comprising administering to the subject an effective amount of an amino acid composition of any one of claims 1 to 20.

27. A method for promoting or assisting exercise performance in a subject, the method comprising administering to the subject an effective amount of an amino acid composition of any one of claims 1 to 20.

28. A method for increasing haemoglobin and/or haematocrit levels in the blood of a subject, the method comprising administering to the subject an effective amount of an amino acid composition of any one of claims 1 to 20.

29. A method for providing nutritive support to the elderly, the method comprising administering to the subject an effective amount of an amino acid composition of any one of claims 1 to 20.

30. A method for reducing fatigue in a subject, the method comprising administering to the subject an effective amount of an amino acid composition of any one of claims 1 to 20.

31. A method according to any one of claims 24 to 30, wherein the subject is a human.

32. The method of claim 31, wherein the composition administered comprises histidine, lysine, serine, ornithine and glycine, and wherein these amino acids comprise between about 50% and 80% of the total weight of amino acids in the composition.

33. A method according to any one of claims 24 to 30, wherein the subject is a horse.

34. The method of claim 33, wherein the composition administered comprises histidine, serine, lysine, glutamine and/or glutamic acid, leucine and aspartic acid, and wherein these amino acids comprise between about 35% and 60% of the total weight of amino acids in the composition.

35. A method for determining a dietary supplement to be administered to a subject, the method comprising:
a) having the subject exercise sufficiently to generate sweat;
b) determining the amino acid composition in said sweat;
c) determining a sweat-facilitated loss of amino acids profile for the subject based on the total amino acid concentration in said sweat, wherein an amino acid concentration of less than about 4,000 μmoles L⁻¹ represents a ‘low’ profile, between about 4,000 and 10,000 μmoles L⁻¹ represents an ‘intermediate’ profile and greater than about 10,000 μmoles L⁻¹ represents a ‘high’ profile;
wherein stratifying the subject as low, intermediate or high sweat-facilitated loss of amino acids profile determines the supplement to be administered, and optionally the quantity or dosage of said supplement to be administered.

36. The method of claim 35, wherein the determination of a sweat-facilitated loss of amino acids profile for the subject further comprises determining individual amino acid concentrations in the sweat, wherein: (i) the ‘low’ profile is represented by serine, glycine, alanine and histidine comprising at least about 50% of the amino acids in the sweat, with serine being the major amino acid component of the sweat; (ii) the ‘intermediate’ profile is represented by ornithine, serine, histidine and glycine comprising at least about 70% of the amino acids in the sweat, with ornithine being the major amino acid component of the sweat; and (iii) the ‘high’ profile is represented by histidine, serine, ornithine and glycine comprising at least about 60% of the amino acids in the sweat, with histidine being the major amino acid component of the sweat.

37. The method of claim 35 or 36, wherein determination of a ‘low’ sweat-facilitated loss of amino acids profile indicates an amino acid supplement for the subject comprising serine, glycine, alanine and histidine, wherein the serine, glycine, alanine and histidine together comprise at least about 60% of the total weight of amino acids in the composition.

38. The method of claim 35 or 36, wherein determination of an ‘intermediate’ sweat-facilitated loss of amino acids profile indicates an amino acid supplement for the subject comprising ornithine, serine, histidine and glycine, wherein the ornithine, serine, histidine and glycine together comprise at least about 64% of the total weight of amino acids in the composition.

39. The method of claim 35 or 36, wherein determination of a ‘high’ sweat-facilitated loss of amino acids profile indicates an amino acid supplement for the subject comprising histidine, serine, ornithine and glycine, wherein the histidine, serine, ornithine and glycine together comprise at least about 76% of the total weight of amino acids in the composition.

40. A method for determining a requirement for dietary supplementation to be administered to a subject, the method comprising:
a) obtaining a plasma sample from the subject; and
b) determining total amino acid composition in said plasma, wherein an amino acid concentration of less than about 2,800 μmoles L⁻¹ represents a ‘low’ operating level of amino acids indicating a need for supplementation.
